(19) United States
(12) Patent Application Publication
(10) Pub. No.: US 2025/0057703 A1
(43) Pub. Date: Feb. 20, 2025

(54) ABSORBENT ARTICLE WITH ABSORBENT CORE STRUCTURE HAVING A SHAPED INNER CORE LAYER

(71) Applicant: The Procter & Gamble Company, Cincinnati, OH (US)

(72) Inventors: Christopher Philip BEWICK-SONNTAG, Cincinnati, OH (US); John David NORCOM, Cincinnati, OH (US); Clint Adam MORROW, Union, KY (US); Miguel Alvaro ROBLES, Wyoming, OH (US)

(21) Appl. No.: 18/804,295

(22) Filed: Aug. 14, 2024

Related U.S. Application Data
(60) Provisional application No. 63/519,340, filed on Aug. 14, 2023.

(51) Int. Cl. A61F 13/535; A61F 13/15; A61F 13/53; A61F 13/538

(52) U.S. Cl. CPC A61F 13/535; A61F 13/538; A61F 2013/15373; A61F 2013/15406; A61F 2013/15422; A61F 2013/15447; A61F 2013/530226; A61F 2013/530343; A61F 2013/530489

(57) ABSTRACT

A disposable absorbent article having a topsheet, a backsheet, and an absorbent core structure disposed therebetween. The absorbent core structure includes an upper nonwoven layer comprising polymer fibers, a lower nonwoven layer comprising polymer fibers, and an inner core layer comprising cellulosic fibers and superabsorbent particles disposed therebetween. The inner core layer is profiled in the longitudinal and lateral directions and is defined by a central absorbent zone and an outer absorbent zone substantially surrounding the central absorbent zone. The basis weight of the central absorbent zone is greater than the basis weight of the outer absorbent zone. The absorbent article has a first average density measured in the central absorbent zone and a second average density measured in the outer absorbent zone. The first and second average density are between about 0.045 g/cm³ and about 0.150 g/cm³, and the second density is within about 0 to about 20% of first density.

### CROSS REFERENCE TO RELATED APPLICATION

This application claims the benefit, under 35 USC 119(e), to U.S. Provisional Patent Application No. 63/519,340 filed on Aug. 14, 2023, which is herein incorporated by reference in its entirety.

### FIELD OF THE INVENTION

The present disclosure relates to absorbent articles comprising an absorbent core structure having a shaped inner core layer.

### BACKGROUND OF THE INVENTION

Absorbent articles are widely used among consumers, e.g., diapers, training pants, feminine pads, adult incontinence pads, etc. Generally, absorbent articles such as these comprise a topsheet and a backsheet, with an absorbent core structure disposed therebetween. These absorbent articles are designed to absorb and retain liquids and other discharges from the human body to prevent body and garment soiling. To absorb fluid effectively without leakage, absorbent articles for menstrual applications should conform closely to a women's genital anatomical shape such that the absorbent article can capture fluid as it exits the labial structure. A common complaint from users of current absorbent articles is the sensation of fluid moving along the body or escaping the article during heavier discharge events. In the case of panty-applied products, the panty and absorbent article often do not fit sufficiently close to the body to address these consumer concerns. Historically, absorbent articles have tried to address this problem by adding more bulk (i.e., absorbent material) to attempt to fill the space between the user's legs. Other approaches have added more absorbent material to the center of the absorbent article (commonly referred to as “more in the middle”) in order to increase the caliper in the central region. This is often accomplished by profiling the cellulose absorbent material in the longitudinal direction or by adding an additional, discrete ellipsoid-shaped absorbent layer.

However, these traditional approaches do not adequately solve the problem of reducing or eliminating the spread of fluid on the body because the additional absorbent material is invariably bulky, stiff, and not shaped to closely and comfortably conform to the wearer's intimate anatomy. In the case of female genitalia, fluid first exits the body internally within the labial vestibule, in particular within the labial minora, prior to exiting through the labial majora. Fluid can exit the labial structure at the top, the bottom, and/or at the sides. Since the labial vestibule is curved, there typically exists a gap between the labia majora such that fluid exiting the labial minora is not captured by simply adding more absorbent material in the central region. Traditional “more in the middle” shapes rest atop of the labial structure and thus forms a bridge across the gap between the labial majora without actually capturing the fluid that resides and is moving from within the labial vestibule outwards. Additionally, typical bulky central regions may actually push the non-raised portions of the absorbent article away from her body, thus causing more gapping on the sides for fluid to spread and be felt by the consumer.

Thus, there is a need for an improved absorbent article that more effectively captures fluid as it exits the labial minora, within the labia majora, across the diverse range of female genitalia shapes and sizes without being stiff and bulky.

### SUMMARY OF THE INVENTION

The present disclosure solves the problem of fluid spreading on the body by providing an absorbent core structure comprising an inner core layer that is shaped to closely fit within and between the upper spaces between the labial majora and to fit in the perinium and at the base of the pubis mons. As described herein, the absorbent article comprises an absorbent core structure comprising a profiled inner core layer which is highly compressible and moldable without being bulky, allowing the absorbent article to comfortably adapt and fit closely to a wide range of female anatomical shapes.

A disposable absorbent article comprises a topsheet; a backsheet; and an absorbent core structure disposed between the topsheet and the backsheet, wherein the absorbent core structure comprises: (a) an upper nonwoven layer comprising polymer fibers and having a basis weight of from about 30 gsm to about 85 gsm; (b) a lower nonwoven layer comprising polymer fibers and having a basis weight of from about 10 gsm to about 40 gsm; and (c) an inner core layer disposed between the upper nonwoven layer and the lower nonwoven layer, wherein the inner core layer comprises from about 50% to about 85% cellulosic fibers, by weight of the inner core layer, and from about 15% to about 50% superabsorbent particles, by weight of the inner core layer; wherein the inner core layer is profiled in the longitudinal and lateral directions, the inner core layer further comprising a central absorbent zone and an outer absorbent zone substantially surrounding the central absorbent zone, wherein the central absorbent zone has a first basis weight and the outer absorbent zone has a second basis weight, wherein the first basis weight is greater than the second basis weight; wherein the absorbent article has a first average density measured in the central absorbent zone and a second average density measured in the outer absorbent zone, wherein the first and second average density are between about 0.045 g/cm³ and about 0.150 g/cm³, and wherein the second density is within about 0 to about 20% of the first density.

A disposable absorbent article comprises a topsheet; a backsheet; and an absorbent core structure disposed between the topsheet and the backsheet, wherein the absorbent core structure comprises: (a) an upper nonwoven layer comprising polymer fibers and having a basis weight of from about 35 gsm to about 85 gsm; (b) a lower nonwoven layer comprising polymer fibers and having a basis weight of from about 10 gsm to about 40 gsm; and (c) an inner core layer disposed between the upper nonwoven layer and the lower nonwoven layer, wherein the inner core layer comprises a mixture of cellulosic fibers and superabsorbent particles; wherein the inner core layer further comprises a central absorbent zone having a first basis weight and an outer absorbent zone having a second basis weight; wherein the outer absorbent zone substantially surrounds the central absorbent zone; wherein the first basis weight is greater than the second basis weight; wherein the inner core layer is a unitary structure; and wherein the absorbent article has a first average density measured in the central absorbent zone and a second average density measured in the outer absorbent zone, wherein the second density is within about 0 to about 20% of first density.

### BRIEF DESCRIPTION OF THE DRAWINGS

FIG. 1 is a perspective view of an absorbent article, according to one or more configurations shown and described herein;

FIG. 2 is a plan view of the absorbent article of FIG. 1, wearer-facing surface facing the viewer, with a portion of the structure being cut-away to more clearly show the construction of the absorbent core structure;

FIG. 3 is a cross-sectional view of FIG. 2 taken along line 3-3 with the topsheet and the backsheet removed to more clearly show the absorbent core structure;

FIG. 4 is a plan view of an example absorbent article, wearer-facing surface facing the viewer, illustrating central absorbent zone and outer absorbent zone configurations, according to one or more configurations shown and described herein;

FIG. 5 is a close up illustration of a structural bond site, according to one or more configurations shown and described herein;

FIG. 6 is a cross-sectional view of the structural bond site of FIG. 5;

FIG. 7A is a cross-sectional view of the absorbent article of FIG. 4 taken along line 7A-7A, illustrating the profile of the inner core layer in the middle region of the absorbent article, according to one or more configurations shown and described herein;

FIG. 7B is a cross-sectional view of the absorbent article of FIG. 4 taken along line 7B-7B, illustrating the profile of the inner core layer in the rear region of the absorbent article, according to one or more configurations shown and described herein;

FIG. 8 is a cross-sectional view of the absorbent article of FIG. 4 taken along line 8-8, illustrating the profile of the inner core layer, according to one or more configurations shown and described herein;

FIG. 9 is a plan view of an example absorbent article, wearer-facing surface facing the viewer, illustrating flex bond channel regions, according to one or more configurations shown and described herein; and

FIGS. 10A-C are a test method arrangement for the Dry CD Ultra Sensitive 3 Point Bending Method.

### DETAILED DESCRIPTION OF THE INVENTION

As used herein “disposable absorbent article” or “absorbent article” shall be used in reference to articles such as diapers, training pants, diaper pants, refastenable pants, adult incontinence pads, adult incontinence pants, feminine hygiene pads, cleaning pads, and the like, each of which are intended to be discarded after use.

As used herein “absorbent core structure” shall be used in reference to the upper nonwoven layer, the lower nonwoven layer, and the inner core layer disposed between the upper nonwoven layer and the lower nonwoven layer. As used herein, “absorbent core structure” does not include any secondary topsheet, topsheet, secondary backsheet, or backsheet of the absorbent article.

As used herein “hydrophilic” and “hydrophobic” have meanings as well established in the art with respect to the contact angle of water on the surface of a material. Thus, a material having a water contact angle of greater than about 90 degrees is considered hydrophobic, and a material having a water contact angle of less than about 90 degrees is considered hydrophilic. Compositions which are hydrophobic, will increase the contact angle of water on the surface of a material while compositions which are hydrophilic will decrease the contact angle of water on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material.

As used herein, the term “filament” refers to any type of continuous strand produced through a spinning process, a meltblowing process, a melt fibrillation or film fibrillation process, or an electrospinning production process, or any other suitable process to make filaments. The term “continuous” within the context of filaments are distinguishable from staple length fibers in that staple length fibers are cut to a specific target length. In contrast, “continuous filaments” are not cut to a predetermined length, instead, they can break at random lengths but are usually much longer than staple length fibers.

As used herein, “machine direction” refers to the direction in which a web flows through an absorbent article converting process. For the sake of brevity, “machine direction” may be referred to as “MD”.

As used herein, “cross machine direction” refers to the direction which is perpendicular to the MD. For the sake of brevity, “cross machine direction” may be referred to as “CD”. “Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

As used herein, “resilient” refers to a material that tends to retain its shape both in the dry and wet states and when subjected to a compression force tends to recover its original, pre-compression shape when such force is removed. In some aspects, the upper and/or lower nonwoven layers described herein may be resilient.

As used herein, “wearer-facing” (sometimes referred to herein as body-facing) and “outward-facing” (sometimes referred to herein as garment-facing) refer respectively to the relative location of an element or a surface of an element or group of elements. “Wearer-facing” implies the element or surface is nearer to the wearer during wear than some other element or surface. “Outward-facing” implies the element or surface is more remote from the wearer during wear than some other element or surface (i.e., element or surface is proximate to the wearer's garments that may be worn over the absorbent article).

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present disclosure relates to disposable absorbent articles comprising a topsheet, a backsheet, and an absorbent core structure which comprises an upper nonwoven layer and a lower nonwoven layer, with an inner core layer comprising a liquid-absorbent material disposed between the upper and lower nonwoven layers. The liquid-absorbent material may comprise a matrix comprising cellulosic fibers and superabsorbent particles, sometimes referred to herein as “fluff/AGM”. The inner core layer may be contained within the nonwoven layers by substantially sealing at least the left side and the right side regions of the upper and lower nonwoven layers at a perimeter seal. In some configurations, the upper and lower nonwoven layers may be joined at a perimeter seal which extends around the entire perimeter of the inner core layer.

The absorbent core structure described herein is configured to compress and recover its original shape (dry or wet) across a range of bodily movements and compressions. The flexibility and/or resiliency of the absorbent core structure results in an absorbent article that comfortably conforms to the wearer's anatomical geometry while efficiently managing the fluid as it exits the body. This can, unexpectedly, be achieved without typical densification stiffening (for wet integrity) by leveraging resilient upper and lower nonwoven layers composed of resilient polymers located above and below the loosely packed liquid-absorbent material of the inner core layer. The absorbent core structure is surprisingly able to carry the structural load and recover shape without physically being stiff or losing the desired structural properties when the absorbent core structure becomes wet. Without being limited by theory, it is believed that wet integrity/shape stability in a cellulose rich absorbent core structure without substantial densification and stiffening may be achieved when select resilient upper and lower nonwovens are positioned above and below the liquid-absorbent material of the inner core layer and are joined to and around the liquid-absorbent material. The upper and lower nonwovens may have sufficient recovery force to carry the liquid-absorbent material back to the original state and/or a stable fiber orientation state following compression. Wrapping or encapsulating a cellulose rich fluff core with a simple cellulose tissue or less resilient nonwoven material may not exhibit sufficient recovery energy to recover shape in-use and particularly when wetted. Structural, wet resilient nonwovens detailed herein may exhibit recovery energies following compression that are sufficient to recover the cellulose rich fiber matrix and are chosen to deliver high compression recovery, with relatively low stiffness, in both dry and wet states. It is believed that suitable absorbent core structures have a low force to compress (less resistance) and the structure is able to recover its shape as the user, in a cyclic fashion, compresses and releases the compressive force with various body movements. To achieve this, the structure should sustain sufficient recovery energy following multiple cyclic compressions. Without sufficient recovery energy, the structure will remain in a compressed bunched state with insufficient force (stored energy) to recover.

The absorbent article described herein further comprises a profiled inner core layer comprising a central absorbent zone and an outer absorbent zone substantially surrounding the central absorbent zone. The central absorbent zone has a basis weight that is greater than the outer absorbent zone, creating a raised 3D structure in the middle region of the absorbent article. As described below, the shape of the inner core layer and/or the central absorbent zone is such that the central absorbent zone may fit and gently conform within and between the upper spaces between the labial majora. The central absorbent zone and the outer absorbent zone are highly compressible without being bulky and thus can fit closely to a wide range of intimate body shapes without discomfort.

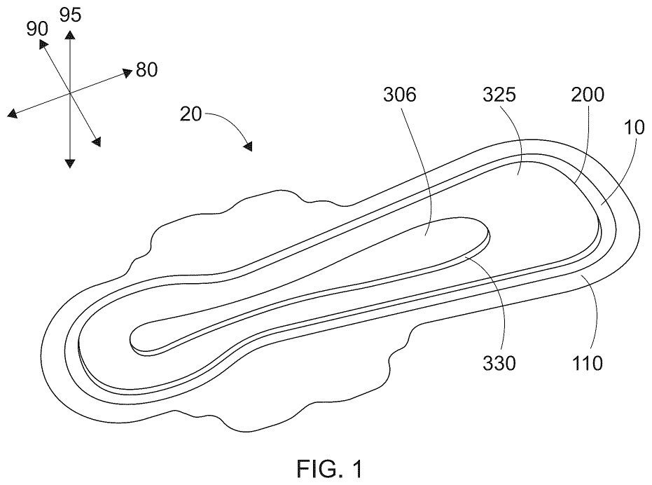

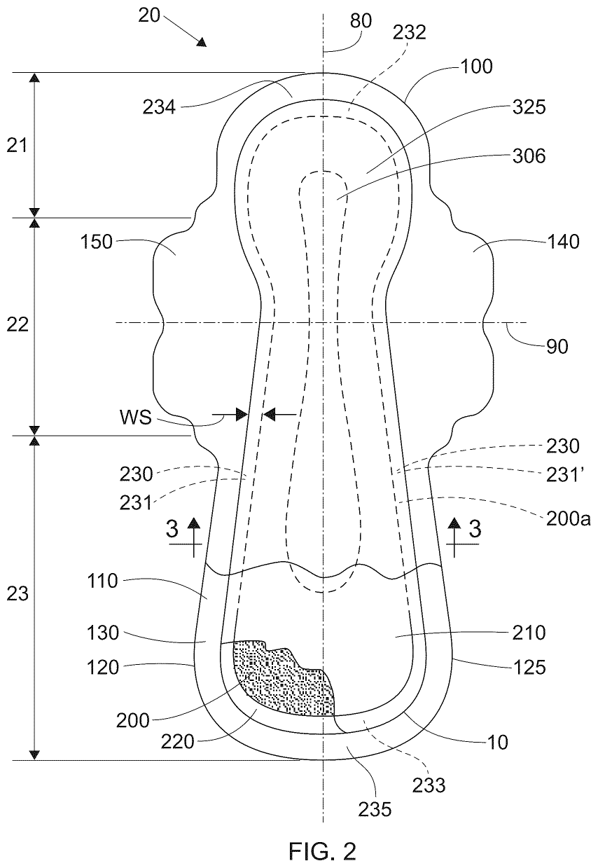

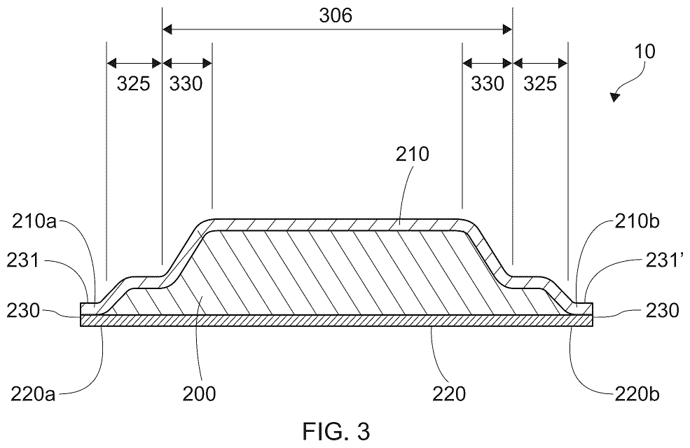

An exemplary absorbent article 20 of the present disclosure is represented in FIG. 1. To provide a frame of reference for the present discussion, the absorbent article 20 of FIG. 1 is shown with a longitudinal axis 80, a lateral axis 90, and a z-direction axis 95 perpendicular to the longitudinal axis 80 and the lateral axis 90. FIG. 2 is a plan view of the absorbent article 20 of FIG. 1, wearer-facing surface facing the viewer, with a portion of the structure being cut-away to more clearly show the construction of the absorbent core structure 10. FIG. 3 is a cross-sectional view taken along lines 3-3 of FIG. 2 with the topsheet 110 and the backsheet 130 removed to more clearly show the absorbent core structure 10.

Referring to FIGS. 1-3, the absorbent article 20 comprises a topsheet 110, a backsheet 130, and the absorbent core structure 10 disposed between the topsheet 110 and the backsheet 130. Absorbent article 20 and absorbent core structure 10 each include a front region 21, a rear region 23, and a middle region 22 disposed intermediate the front region 21 and the rear region 23.

The absorbent core structure 10 may comprise an upper nonwoven layer 210 and a lower nonwoven layer 220 (also referred to herein collectively as upper and lower nonwoven layers or upper and lower nonwovens) and an inner core layer 200 disposed between the upper nonwoven layer 210 and the lower nonwoven layer 220. The inner core layer 200 may comprise a liquid-absorbent material, such as, for example, cellulosic fibers and superabsorbent particles. In some configurations, the liquid-absorbent material may be uniformly distributed. In some configurations, the liquid-absorbent material may be present discontinuously within the absorbent core structure 10, for example, as individual pockets or stripes of liquid-absorbent material separated from each other by material-free areas. In some configurations, the absorbent core structure 10 may have a non-rectangular perimeter. In particular, the absorbent core may be shaped to define a tapering along its width towards the middle region of the absorbent core structure. The absorbent core structure may conform to a wearer's inner thigh geometry, such as, for example, an hourglass shape, an offset hourglass shape (one end is wider than an opposite end and a narrowed mid-section between the ends), a bicycle seat shape (one end and central portion are narrower than the second end), an oval, or a trapezoid shape.

The inner core layer 200 may comprise a central absorbent zone 306 that extends in the longitudinal direction of the absorbent article from the front region 21 to the rear region 23, and an outer absorbent zone 325 that substantially surrounds the central absorbent zone 306. The central absorbent zone may comprise a transition zone 330 that extends around the periphery of the central absorbent zone where the basis weight of the inner core layer gradually decreases. The inner core layer 200 may be profiled in the longitudinal and lateral direction such that the basis weight of the inner core layer is greater in the central absorbent zone 306 than in the outer absorbent zone 325.

In some configurations, the absorbent article 20 may comprise the following structure (from a wearer-facing surface to an outward-facing surface): a topsheet 110, an upper nonwoven layer 210, an inner core layer 200, a lower nonwoven layer 220, and a backsheet 130. In some aspects, the topsheet 110 may be in direct contact with the upper nonwoven layer 210, the upper nonwoven layer 210 may be in direct contact with the inner core layer 200, and/or the inner core layer 200 may be in direct contact with the lower nonwoven layer 220. By “direct contact”, it is meant that there is no further intermediate component layer between the respective layer in direct contact thereto. It is however not excluded that an adhesive material may be disposed between at least a portion of the layers described above.

Upper nonwoven layer 210 may comprise a left side region 210a and a right side region 210b, and lower nonwoven layer 220 may comprise a left side region 220a and a right side region 220b. The upper and lower nonwoven layers 210, 220 may extend outwardly from an inner core layer perimeter 200a and may be joined together with glue or other conventional bonding methods including, but not limited to, ultrasonic bonding, fusion bonding, crimping, and combinations thereof, to form a perimeter seal 230. In some configurations, the entire inner core layer 200 may be located inboard of the perimeter seal 230. The perimeter seal 230 may help to seal the liquid-absorbent material of the inner core layer 200 inside the upper and lower nonwoven layers 210, 220. Perimeter seal 230 may comprise at least a first lateral seal region 231 and a second lateral seal region 231′. In some configurations, perimeter seal 230 may further comprise a front perimeter seal region 232 and/or a back perimeter seal region 233. In some configurations, the perimeter seal 230 may extend around the entire inner core layer perimeter 200a. In some configurations, the perimeter seal 230 may extend partially around the inner core layer perimeter 200a.

In some configurations, the inner core layer 200 may be contained within the upper nonwoven layer 210 and the lower nonwoven layer 220 by substantially sealing at least a left side region 210a, 220a and a right side region 210b, 220b of the upper nonwoven layer 210 and the lower nonwoven layer 220. In some configurations, the inner core layer 200 may be contained within the upper nonwoven layer 210 and the lower nonwoven layer 220 by sealing at least a portion of the left side region 210a, 220a and the right side region 210b, 220b of the upper nonwoven layer 210 and the lower nonwoven layer 220. Without being limited by theory, it is believed that resilient nonwoven layers comprising polymer fibers may hold their shape and resist plasticizing when wet when attached to the inner core layer through the application of a core construction adhesive that is applied either directly to the inner core layer or the resilient nonwoven layer(s) via a conventional spray coating application chosen to achieve a bond but not disrupt the flow of fluid to the inner core layer. The perimeter seal 230 may be positioned in at least the middle region 22 of the absorbent article 20 and/or the absorbent core structure 10. It is believed that the middle region 22 (located between the wearer's thighs during use) may be subjected to the most frequent and/or highest forces during use. It was found that the presence of at least a partial perimeter seal at a left side region and a right side region of the upper nonwoven layer and the lower nonwoven layer external to the inner core layer may help to ensure the upper and lower nonwovens maintain their structural function during physical deformations without separating, limiting any potential integrity and bunching issues. In addition, a perimeter seal may allow for any excess nonwoven material to be removed in order to enable an absorbent core structure to be shaped to conform to inner thigh geometry.

The perimeter seal 230 may have a seal width WS of between about 1 mm and about 10 mm, or between about 2 mm and about 8 mm, or between about 3 mm and 6 mm. The seal width WS may be uniform or may vary about the perimeter of the inner core layer. In some configurations, the absorbent article 20 may also comprise a front end seal 234 positioned in the front region 21 of the absorbent article and a back end seal 235 positioned in a rear region 23 of the absorbent article. The front end seal 234 and/or back end seal 235 may seal the topsheet, upper nonwoven layer, lower nonwoven layer, and the backsheet together. In some configuration, the front end seal 234 and/or the back end seal 235 may seal the topsheet and the backsheet. In some configurations, the front end seal 234 and/or the back end seal 235 may be a crimp seal.

In some configurations, the upper and lower nonwoven layers 210, 220 may be discrete materials that can be cut to approximately the size and shape of the inner core layer 200 so as to fit between the topsheet 110 and backsheet 130 but may not extend substantially into either the front end seal 234 or the back end seal 235. In some configurations, the inner core layer 200, upper nonwoven layer 210 and/or lower nonwoven layer 220 may be shaped, meaning it is non-rectangular. In some configurations, the upper and/or lower nonwoven layers 210, 220 may extend from the front edge of the absorbent article, through the front end seal 234 and the back end seal 235, to the back edge of the absorbent article.

As shown in FIG. 2, the absorbent article 20 may further comprise a chassis 100 comprising the absorbent core structure 10. The absorbent core structure 10 and/or the inner core layer 200 may be shaped. Side edges 120 and 125 of the absorbent article 20 may follow the general contour of the absorbent core structure 10 and/or the inner core layer 200. So, for example, where the absorbent core structure 10 has an hourglass shape, the side edges 120, 125 of the absorbent article 20 may be arranged in an hourglass shape as well. However, forms are contemplated where the side edges 120 and 125 are generally straight or slightly curved such that they do not follow the contour of the absorbent core structure. In some configurations, the absorbent article 20 may be symmetric about the longitudinal centerline 80 or asymmetric about the longitudinal centerline 80. Similarly, the absorbent article 20 may be symmetric about the lateral centerline 90 or asymmetric about the lateral centerline 90.

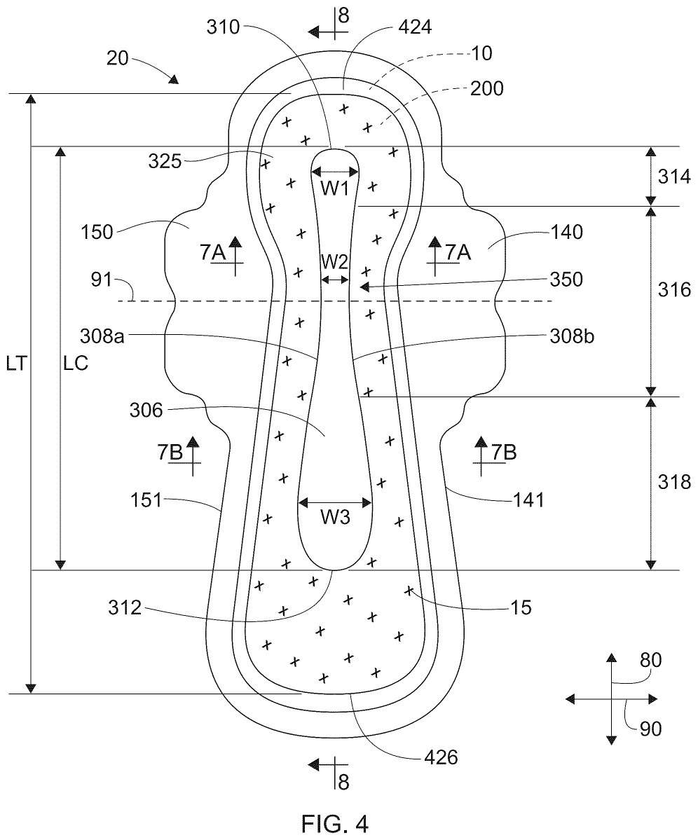

As previously mentioned, the absorbent article 20 may comprise an inner core layer 200 having a profiled distribution of liquid-absorbent material in the longitudinal and lateral direction. FIG. 4 is a plan view of an absorbent article 20 illustrating inner core layer 200 size and shape features. The inner core layer 200 may comprise a central absorbent zone 306 having a first basis weight and an outer absorbent zone 325 having a second basis weight. The outer absorbent zone 325 substantially surrounds the central absorbent zone 306. The first basis weight of the central absorbent zone 306 may be greater than the second basis weight of the outer absorbent zone 325. In some configurations, the first basis weight and the second basis weight may differ by about 20% to about 100%. The first basis weight may be from about 220 gsm to about 450 gsm, or from about 300 gsm to about 425 gsm, as measured according to the Inner Core Layer Basis Weight Method. The second basis weight may be from about 150 gsm to about 320 gsm, or from about 200 gsm to about 300 gsm as measured according to the Inner Core Layer Basis Weight Method.

It is to be appreciated that the increase in basis weight in the central absorbent zone 306 is due to additional liquid-absorbent material in the inner core layer 200 positioned in the central absorbent zone 306 relative to the outer absorbent zone 325. The inner core layer 200 described herein may be a unitary structure. As used herein, “unitary structure” means that the inner core layer 200 is continuous and is constructed from essentially one type of material, this being essentially the same material, or essentially the same combination of two or more materials throughout the inner core layer 200. Variations in density and concentration of the material may occur, but these are limited to those which may be obtained without incorporation of regions which have been formed separately and then physically joined together. For example, when the inner core layer 200 comprises a liquid absorbent material, e.g., cellulosic fibers and superabsorbent polymers, the relative concentrations of superabsorbent particles and cellulosic fibers may be different in different parts of the inner core layer 200. However, when the construction is unitary, the inner core layer 200 does not, for instance, include layers or laminates of a different composition. Likewise, variations in the density or concentration of various components across the longitudinal direction, the lateral direction, or the thickness direction of the inner core layer 200 may occur, yet the inner core layer 200 should not include areas or layers of different composition which are formed separately and later joined together or areas of the same or different material that are physically separated by areas of substantially no basis weight of liquid absorbent material.

The absorbent article 20 may have varying caliper in the longitudinal and lateral directions (e.g., profiled so as to have a higher caliper in the center). In some configurations, the absorbent article 20 may have a first caliper measured in the central absorbent zone 306, and a second caliper measured in the outer absorbent zone 325. In some configurations, the first caliper may be from about 2.5 mm to about 6 mm, and the second caliper may be from about 1.0 mm to about 3.0 mm, as measured according to the Absorbent Article Caliper, Basis Weight and Density Method. In some configurations, the ratio of the first caliper to the second caliper may be from about 1.2 to about 2.5.

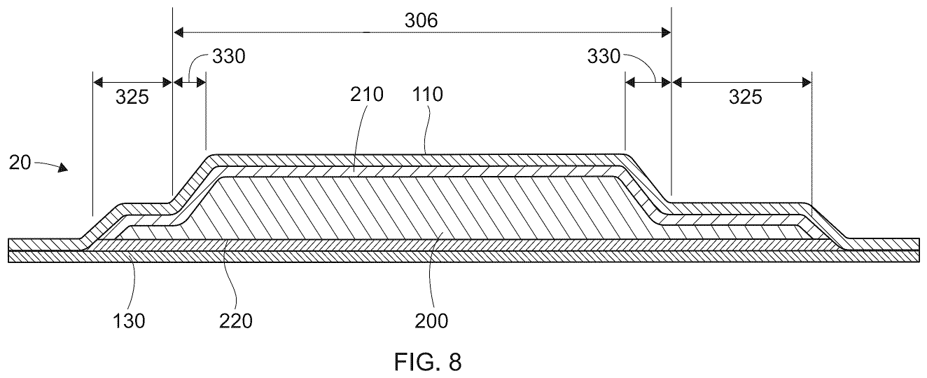

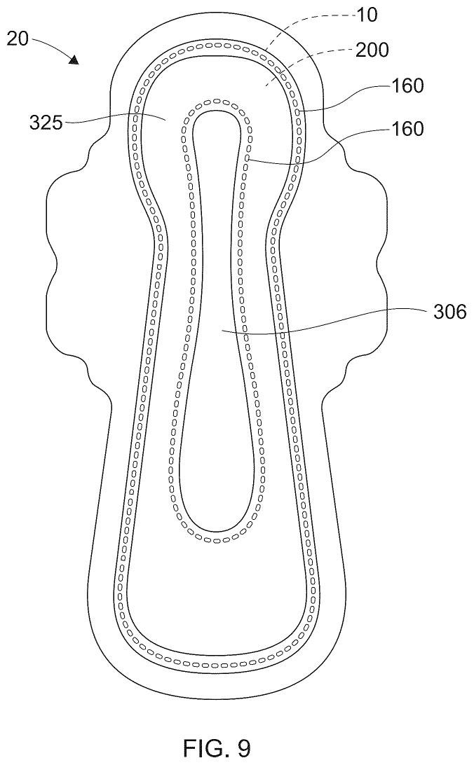

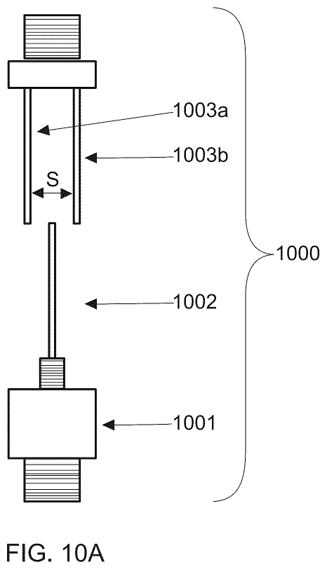

As described in more detail below, the inner core layer may have a non-rectangular perimeter. In particular, the inner core layer 200 be shaped such that the liquid-absorbent material in the central absorbent zone may define a tapering along its width towards the middle region such that it creates a 3D shape that is better able to fit within and between the upper spaces between the labial majora and to fit in the perinium and at the base of the pubis mons. FIG. 7A is a cross section view of the absorbent article 20 of FIG. 4 taken along lines 7A-7A illustrating inner core layer size and shape features in the middle region 22 of the absorbent article. FIG. 7B is a cross section view of the absorbent article 20 of FIG. 4 taken along lines 7B-7B illustrating inner core layer size and shape features in the rear region 23 of the absorbent article. FIG. 8 is a cross section view of the absorbent article 20 of FIG. 4 taken along lines 8-8 illustrating inner core layer size and shape features.

As shown in FIGS. 7A-8, the central absorbent zone 306 may comprise a transition zone 330 wherein the basis weight of the inner core layer gradually decreases. The upper nonwoven layer 210 is not in contact with the lower nonwoven layer 220 in the transition zone 330 as liquid-absorbent material of the inner core layer is disposed therebetween. In some configurations, the transition zone 330 may have a width (TZ) of from about 1 mm to about 5 mm, or from about 2 mm to about 3 mm. The width of the transition zone may be measured from images obtained from microCT and analyzed by image analysis. Without being limited by theory, it is believed that a transition zone having the above described width may help to create the desired 3D shape of the central absorbent zone and allow it to fit closely to the body.

As shown in FIG. 4, the central absorbent zone 306 may be shaped, meaning that it is non-rectangular. The central absorbent zone 306 may be shaped to gently sit between and within the external gap between the labia majora while gently contacting the external part of the labial minora where blood exits (without penetrating internally between the labia minora). Suitable shapes for the central absorbent zone include, but are not limited to, an hourglass shape, an offset hourglass shape (one end is wider than an opposite end and a narrowed mid-section between the ends), or a bicycle seat shape (one end and central portion are narrower than the second end).

In some configurations, the central absorbent zone 306 may define a perimeter comprising a pair of inwardly concave longitudinal side edges 308a, 308b, an outwardly convex front edge 310, and an outwardly convex rear edge 312. The central absorbent zone 306 may comprise a front region 314 having a first lateral width W1, a rear region 318 having a third lateral width W3, and a middle region 316 positioned therebetween having a second lateral width W2. In some configurations, the second lateral width W2 may be less than the first lateral width W1 and/or the third lateral width W3. In some configurations, the third lateral width W3 may be greater than the first lateral width W1 and the second lateral width W2. The first lateral width W1 may be from about 20 mm to about 35 mm, or from about 22 mm to about 30 mm, as measured from the outer most point of the first inwardly concave longitudinal side edge to the outer most point of the second inwardly concave longitudinal side edge in front region 314. The second lateral width W2 is from about 10 mm to about 20 mm, or from about 12 mm to about 15 mm, as measured from the inner most point of the first inwardly concave longitudinal side edge to the inner most point of the second inwardly concave longitudinal side edge in middle region 316. The third lateral width W3 may be from about 30 mm to about 45 mm, or from about 32 mm to about 40 mm, as measured from the outer most point of the first inwardly concave longitudinal side edge to the outer most point of the second inwardly concave longitudinal side edge in rear region 318. In some configurations, the second lateral width W2 is from about 20% to about 40% of a minimum lateral width of the inner core layer.

In some configurations, the central absorbent zone 306 may have a longitudinal length LC of from about 115 mm to about 200 mm, or from about 125 mm to about 195 mm, as measured from the outermost point of the outwardly convex front edge 310 to the outermost point of the outwardly convex rear edge 312. In some configurations, the central absorbent zone 306 may have a longitudinal length LC of from about 50% to about 75% of an inner core layer longitudinal length LT.

Without being limited by theory, it is believed that the shape of the central absorbent zone allows the central absorbent zone to more effectively capture fluid as it exits the labial minora, within the labia majora across a wide range of female genitalia shapes and sizes. It is believed that in order to effectively fit and gently conform within and between the upper spaces between the labial majora, the shape of the central absorbent zone should be narrow, e.g., a lateral width in the 10-20 mm range, and the narrow portion should be of a corresponding narrowed length, e.g., around 50-80 mm long. In addition, the central absorbent zone should be of a generally concave shape that allows for intimate contact with the body to avoid fluid spreading in a lateral direction, but also be wider in both the front and rear regions so as to gently fit in the perinium and at the base of the pubis mons where natural depressions of the body can allow fluid to bypass a pad and spread on the body.

In some configurations, the outwardly convex front edge 310 of the central absorbent zone 306 may be positioned a distance of from about 25 mm to about 45 mm from a front edge 424 of the inner core layer 200. In some configurations, the outwardly convex rear edge 312 of the central absorbent zone 306 may be positioned a distance of from about 25 mm to about 85 mm from a rear edge 426 of the inner core layer 200.

It may also be described that the shape of the central absorbent zone 306 may taper as it extends from the front region of the absorbent article to the middle region, defining a narrow portion 350 between the inwardly concave longitudinal side edges. The narrow portion 350 of the central absorbent zone may have a width at its narrowest point that is positioned forward of or coterminous with a lateral centerline 91 of the first and second wing portions 140, 150. The narrow portion 350 of the central absorbent zone 306 may be positioned in the middle region 22 of the absorbent article 20. The narrow portion may have a lateral width of from about 10 mm to about 20 mm. The narrow portion may have a lateral width that is from about 20% to about 40% of a minimum lateral width of the inner core layer WC.

In some configurations, the absorbent core structure 10 and/or the inner core layer 200 may be shaped to substantially follow the shape of the central absorbent zone 306.

Although the central absorbent zone 306, outer absorbent zone 325, and the transition zone 330 have been discussed with reference to the inner core layer 200, the description for the different zones may also apply to the absorbent article 20. The basis weight of the absorbent article in the central absorbent zone may be from about 300 gsm to about 500 gsm, as measured according to the Absorbent Article Caliper, Basis Weight and Density Method. The basis weight of the absorbent article in the outer absorbent zone may be from about 200 gsm to about 400 gsm, as measured according to the Absorbent Article Caliper, Basis Weight and Density Method.

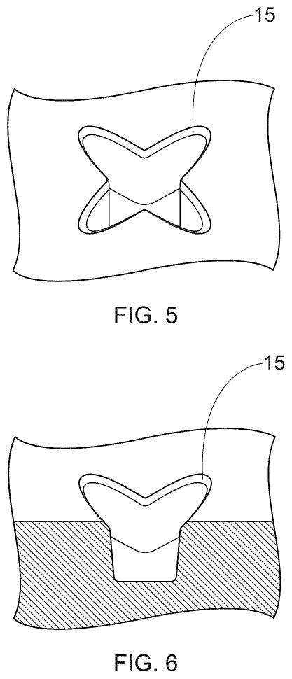

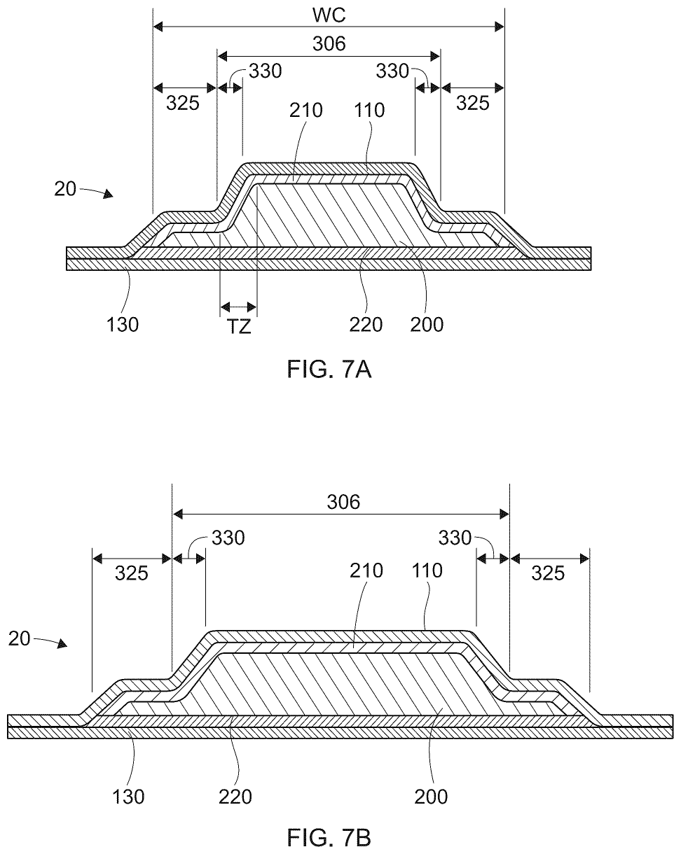

In some configurations, as shown in FIG. 4, the absorbent article 20 may further comprise a plurality of structural bond sites 15. FIGS. 5 and 6 show illustrations of example structural bond sites 15. FIG. 5 is a close up illustration of an example structural bond site 15. FIG. 6 is a cross-section view of the structural bond site 15 of FIG. 5. The structural bond sites 15 may be symmetric and/or asymmetrical and may be any shape including, but not limited to, circles, ovals, hearts, diamonds, triangles, squares, stars, and/or X shapes. While the shape of the structural bond sites may be any shape, suitable shapes may be more detailed shapes such as asymmetrical shapes (versus simple dots). The structural bond sites 15 may be on the absorbent article and/or on the absorbent core structure. In some configurations, the structural bond sites may have a bond area of from about 2 mm² to about 5 mm². In some configurations, the total structural bond area may be from about 0.5% to about 5%, or from about 0.75% to about 4.5%, or from about 1% to about 4% of the absorbent core structure, as measured according to the Structural Bond Sites Pattern Spacing and Area Measurement Method. In some configurations, the total structural bond area may be from about 1% to about 4% of absorbent article as measured according to the Structural Bond Sites Pattern Spacing and Area Measurement Method. The average distance between the structural bond sites may be from about 10 mm to about 32 mm. In some configurations, the average distance between the structural bond sites may be greater than about 20 mm. In some configurations, the structural bond sites may have a maximum width of from about 1 mm to about 6 mm, or from about 1.5 mm to about 5 mm, or from about 2 mm to about 4 mm. Without being limited by theory, it is believed that the average distance between structural bond sites and/or the size of the structural bond sites may help to maintain the structural integrity of the absorbent core structure without creating an undesirable stiffness that may inhibit the ability of the absorbent article to conform to the body.

In some configurations, the structural bond sites may be distributed across the absorbent article and/or absorbent core structure or they may be clustered in regions of the absorbent article and/or absorbent core structure. In some configurations, the structural bond sites may be clustered in the middle region 22 of the absorbent article 20 and/or absorbent core structure 10. In some configurations, the middle region 22 of the absorbent article 20 and/or absorbent core structure 10 may be substantially free from structural bond sites and may be surrounded by an area of structural bond sites and/or embossing. In some configurations, as shown in FIG. 4, the outer absorbent zone 325 may comprise a plurality of structural bond sites 15 and the central absorbent zone 306 may be substantially free from structural bond sites 15.

In some configurations, the structural bond sites 15 may join the topsheet 110, the upper nonwoven layer 210, the absorbent core structure 10, and the lower nonwoven layer 220. In some configurations, the structural bond sites 15 may join the upper nonwoven layer 210, the absorbent core structure 10, and the lower nonwoven layer 220. Absorbent articles 20 and/or absorbent core structures 10 may comprise an upper nonwoven layer 210 and a lower nonwoven layer 220 that are closer together in the Z-direction at the structural bond sites 15 but are not melted together. Since these structural bond sites are not melted together, they may not be permanent in nature and rather may intermingle the materials within the structural bond site. In some configurations, the structural bond sites 15 may be substantially free of fusion bonds.

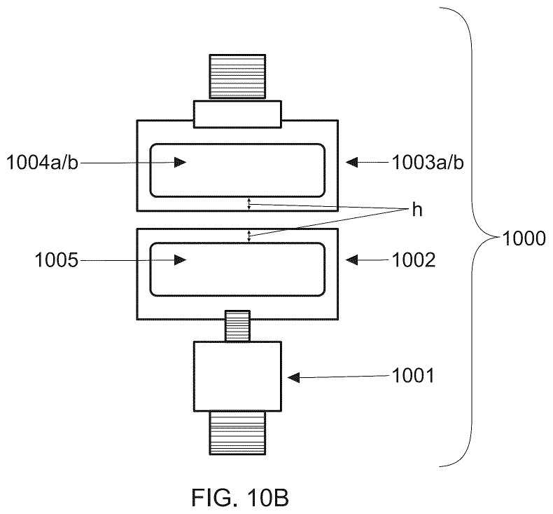

As shown in FIG. 9, the absorbent article 20 may also comprise one or more flex bond channel regions 160. In some configurations, at least one flex bond channel region 160 may be positioned between the central absorbent zone 306 and the outer absorbent zone 325. In some configurations, the flex bond channel region 160 may be positioned in the transition zone 330 or in the outer absorbent zone 325. In some configurations, the flex bond channel region 160 may be positioned adjacent to the transition zone 330 in the outer absorbent zone 325. In some configurations, the flex bond channel region 160 may be positioned about 1 mm to about 10 mm, or from about 3 mm to about 6 mm, outboard of the outermost edge of the transition zone 330. The flex bond channel regions 160 may be a continuous depression and/or a series of individually compressed, closely spaced embossments.

The central absorbent zone 306 and/or the outer absorbent zone 325 may each exhibit an Energy of Z-Compression of from about 3.0 to about 8.0 N·mm as measured according to the Z-Compression Method. In some configurations, the Energy of Z-Compression of the central absorbent zone may be substantially the same as the Energy of Z-Compression of the outer absorbent zone. Without being limited by theory, it is believed that by having an Energy of Z-Compression of from about 3.0 to about 8.0 N·mm provides a high level of compressibility and conformability to gently fit within the available space between the upper space between the labial majora without discomfort or excessive bulk that could disrupt the ability to fit and adapt to this location without moving too deeply within the labia majora or become interlabial in nature.

The absorbent article may exhibit a MD Bending Modulus measured in the central absorbent zone 306 and the outer absorbent zone 325 of from about 0.03 to about 0.18 N/mm², or from about 0.03 to about 0.15 N/mm², as measured according to the Dry MD 3 Point Bend Method.

In some configurations, the MD Bending Modulus of the central absorbent zone 306 may be substantially the same as the MD Bending Modulus of the outer absorbent zone 325. Without being limited by theory, it is believed that by having a MD Bending Modulus of from about 0.03 to about 0.18 N/mm² in the central absorbent zone and the outer absorbent zone helps to provide both the integrity and the comfort of the narrow central absorbent zone. If the MD Bending Modulus is too low, the material risks being too flimsy and rather than residing within the upper reaches between the labial majora it is likely to simply buckle over or bend away from the opening. If the MD modulus is too high, the ability of the narrow central absorbent zone to comfortably fit and conform within the upper space between the labial majora can be compromised or even result in the central absorbent zone being unable to remain within the upper space between the labial majora as the wearer moves about her daily routine.

The average density of the absorbent article measured in the central absorbent zone 306 and the outer absorbent zone 325 may be from about 0.045 g/cm³ and about 0.150 g/cm³ as measured according to the Absorbent Article Caliper, Basis Weight, and Density Method. In some configurations, the average density of the absorbent article measured in the central absorbent zone 306 and the outer absorbent zone 325 may be substantially similar. In some configurations, the average density of the absorbent article measured in the outer absorbent zone 325 may be within about 0 to about 20%, or about 0 to about 18%, or about 0 to about 10%, or about 0 to about 5%, of the average density measured in the central absorbent zone.

Suitable upper nonwoven layers may have a basis weight of from about 30 gsm to about 85 gsm, or from about 35 gsm to about 70 gsm, or from about 40 to about 60 gsm. The upper nonwoven layer may have a Tensile Stiffness of from about 0.1 N/mm to about 2.2 N/mm, or from about 0.3 N/mm to about 1.6 N/mm as measured according to the CD Cyclic Elongation to 3% Strain Method. The upper nonwoven layer may have a Strain to Break of greater than about 10%, or from about 10% to about 50%, or from about 20% to about 40%, as measured according to the Strain to Break Method. The upper nonwoven layer may have a Permanent Strain of about 0.005 to about 0.013 mm/mm, or from 0.005 to about 0.0090 mm/mm, as measured according to the CD Cyclic Elongation to 3% Strain Method. The upper nonwoven layer may have Thickness at 7 g/cm² pressure of from about 0.3 mm to about 1.3 mm and/or a Thickness at 70 g/cm² pressure of from about 0.2 mm to about 0.7 mm, as measured according to the Nonwoven Thickness—Pressure Method.

Suitable lower nonwoven layers may have a basis weight of from about 10 to about 40 gsm, or from about 15 to about 20 gsm. The lower nonwoven layer may have a Tensile Stiffness of from about 0.2 N/mm to about 2.0 N/mm, as measured according to the CD Cyclic Elongation to 3% Strain Method. The lower nonwoven layer may have a Strain to Break of greater than about 10%, or from about 10% to about 50%, or from about 20% to about 40%, as measured according to the Strain to Break Method. The lower nonwoven layer may have a Permanent Strain of about 0.005 to about 0.018 mm/mm, as measured according to the CD Cyclic Elongation to 3% Strain Method. The lower nonwoven layer may have a Thickness at 7 g/cm² pressure of from about 0.1 mm to about 1.3 mm, as measured according to the Nonwoven Thickness—Pressure Method.

The upper and lower nonwoven layers may comprise polymer fibers. Suitable upper and lower nonwoven fibers may be selected from PET (polyethylene terephthalate), PP (polypropylene), a BiCo (Bicomponent fiber) selected from PE/PP (PE sheath and PP core) and/or PE/PET (PE sheath PET core), PLA (polylactic acid), and combinations thereof.

Suitable upper nonwovens may comprise from about 60 to about 100%, or from about 70% to about 100% synthetic fibers, or from about 0 to about 40%, or from about 0 to about 30% regenerated cellulosic fibers, such as rayon and/or viscose.

The upper nonwoven layer may comprise fibers having a staple length of greater than about 10 mm, or greater than about 25 mm, or from about 10 mm to about 100 mm, or from about 20 mm to about 75 mm, or from about 25 mm to about 50 mm. The upper nonwoven layer may comprise fibers having a fiber diameter of from about 1.3 DTex to about 10.0 DTex, alternatively from about 1.3 DTex to about 6.0 DTex, alternatively from about 2.0 DTex to about 5.0 DTex. Without being limited by theory, it is believed that if the fibers of the upper nonwoven layer are less than about 1.3 Dtex, there may be insufficient air flow through the material during manufacturing.

In some configurations, the upper nonwoven layer may comprise a blend of staple fibers. When the upper nonwoven layer comprises a blend of staple fibers, the blend of fibers preferably comprises 30% or less of fibers having a fiber diameter of 1.3 Dtex and/or 30% or less of fibers having a fiber diameter of 10.0 Dtex. In some configurations, the upper nonwoven layer may comprise fibers, wherein the fibers are a blend of staple fibers having an average fiber diameter of from about 2.0 DTex to about 8.0 DTex. Without being limited by theory, it is believed that fibers having an average fiber diameter of from about 2.0 Dtex to about 8.0 Dtex will help to enable sufficient air flow through the material during manufacturing of the absorbent core structure.

The lower nonwoven layer may comprise fibers having a length of greater than about 10 mm, or greater than about 25 mm, or from about 10 mm to about 100 mm, or from about 20 mm to about 75 mm, or from about 25 mm to about 50 mm. In some configurations, the lower nonwoven layer may comprise continuous fibers. The lower nonwoven layer may comprise fibers having a fiber diameter of from about 1.3 DTex to about 5.0 DTex, or from about 1.3 DTex to about 3.3 DTex, or from about 1.3 DTex to about 2.2 DTex, or from about 2.0 DTex to about 10.0 DTex. In some configurations, the lower nonwoven layer may comprise fibers, wherein the fibers are a blend of fibers having a fiber diameter of from about 0.1 DTex to about 6.0 DTex.

In some configurations, upper nonwoven layer may comprise a blend of fibers, wherein at least a portion of the fibers have a diameter of from about 2.0 DTex to about 10 DTex and the lower nonwoven layer may comprise a blend of fibers, wherein at least a portion of the fibers have a diameter of from about 1.3 DTex to about 5 DTex. In some configurations, the upper nonwoven layer may comprise a blend of fibers, wherein at least a portion of the fibers have a diameter of from about 1.3 DTex to about 2.2 DTex and the lower nonwoven layer may comprise a blend of fibers, wherein the blend of fibers have a diameter of from about 1.3 DTex to about 5 DTex.

Suitable upper and/or lower nonwoven layer materials may bend and recover their original shape following the bending force. Flimsy or highly flexible materials readily bend at low peak force (load) and with low bending energy. Unsuitable materials, while readily bending, do not have sufficient recovery energy and so retain a deformed, bent state because of insufficient recovery energy. Suitable materials have sufficient energy to recover their initial pre-bent state. The materials with sufficient bending recovery energy may be considered resilient upper and lower nonwoven layers. Particularly suitable upper nonwoven layers may have a Dry Recovery Energy of greater than about 0.03 N*mmm, or from about 0.03 N*mm to about 1 N*mm, or from about 0.04 N*mm to about 0.5 N*mm. Particularly suitable upper nonwoven layers may have a Dry Bending Energy of less than about 1.6 N*mm, or less than about 1.1 N*mm.

As noted above, the upper and lower nonwovens may include polymer fibers. Polymer fibers may be included to help provide structural integrity to the upper and lower nonwovens. The polymer fibers may help increase structural integrity of the upper and lower nonwovens in both a machine direction (MD) and in a cross-machine direction (CD), which may facilitate web manipulation during processing of the upper and lower nonwovens for incorporation into a pad.

Polymer fibers of any suitable composition may be selected. Some examples of suitable polymer fibers may include bi-component fibers comprising polyethylene (PE) and polyethylene terephthalate (PET) components or polyethylene terephthalate and co-polyethylene terephthalate components. The components of the bi-component fiber may be arranged in a sheath-core configuration, a side-by-side configuration, an eccentric sheath-core configuration, a trilobal arrangement, or any other desired configuration. In some configurations, the polymer fibers may include bi-component fibers having PE/PET components arranged in a concentric, sheath-core configuration, wherein the polyethylene component forms the sheath.

While other materials may be useful in creating a resilient structure, it is believed that the stiffness of a PET core component in a sheath-core fiber configuration may be useful for imparting resilience to the upper and lower nonwovens. In synergistic combination, a PE sheath component, having a lower melting temperature than the PET core component, may be utilized to provide inter-fiber melt/fusion bonding, effected via heat treatment of the precursor batt. This can help provide tensile strength to the web in both the MD and CD. Such inter-fiber bonds may serve to reduce fiber-to-fiber sliding, and thereby further contribute to imparting shape stability and resiliency to the material even when it is wetted.

Where a relatively higher weight fraction of polymer fibers is included, more connections within the structure may be created via heat treatment. However, too many connection points may impart greater stiffness to the upper and lower nonwovens than may be desirable. For this reason, selecting the weight fraction of the polymer fibers may involve prioritizing and balancing competing needs for stiffness and softness in the upper and lower nonwovens.

As noted above, the upper and lower nonwovens may additionally include polymer fibers which increase resiliency of the upper and lower nonwovens. The resilient polymer fibers may help the upper and lower nonwovens maintain permeability and compression recovery. In some configurations, the upper and lower nonwovens may comprise resilient polymer fibers having varying cross sections, e.g., round and hollow spiral, and/or may comprise resilient fibers having varying sizes.

The polymer fibers may be resilient and may be spun from any suitable thermoplastic resin, such as polypropylene (PP), polyethylene terephthalate (PET), or other suitable thermoplastics known in the art. The average staple length of the resilient polymer fibers may be selected to be in the range of greater than about 10 mm, from about 20 mm to about 100 mm, or about 30 mm to about 50 mm, or about 35 mm to about 50 mm. The resilient polymer fibers may have any suitable structure or shape. For example, the resilient polymer fibers may be round or have other shapes, such as spiral, scalloped oval, trilobal, scalloped ribbon, and so forth. Further, the resilient polymer fibers may be solid, hollow, or multi-hollow. The resilient polymer fibers may be solid and round in shape. In other suitable examples, resilient polymer fibers may include polyester/co-extruded polyester fibers. Other suitable examples of resilient polymer fibers may include bi-component fibers such as polyethylene/polypropylene, polyethylene/polyethylene terephthalate, polypropylene/polyethylene terephthalate bicomponent fibers. These bi-component fibers may have a sheath/core configuration.

The resilient polymer fibers may be polyethylene terephthalate (PET) fibers, or other suitable non-cellulosic fibers known in the art. PET fibers may be imparted with any suitable structure or shape. For example, the PET fibers may be round or have other shapes, such as spiral, scalloped oval, trilobal, scalloped ribbon, hollow spiral, and so forth. The PET fibers may be solid, hollow or multi-hollow. In one particular example, PET fibers may be hollow in cross section and have a curl or spiral configuration along their lengths. Optionally, the resilient polymer fibers may be spiral-crimped or flat-crimped. The resilient polymer fibers may have an average crimp count of about 4 to about 12 crimps per inch (cpi), or about 4 to about 8 cpi, or about 5 to about 7 cpi, or about 9 to about 10 cpi. Particular non-limiting examples of resilient polymer fibers may be obtained from Wellman, Inc. (Ireland) under the trade designations H1311 and T5974. Other examples of suitable resilient polymer fibers are disclosed in U.S. Pat. No. 7,767,598.

The stiffening polymer fibers and resilient polymer fibers should be carefully selected. For example, while the constituent polymers forming the stiffening polymer fibers and the resilient polymer fibers may have similarities, resilient polymer fiber composition should be selected such that their constituents' melting temperature(s) is/are higher than that of the bondable components of the stiffening polymer fibers. Otherwise, during heat treatment, resilient polymer fibers could bond to stiffening polymer fibers and vice versa, and thereby an overly rigid structure. To avoid this risk where the stiffening polymer fibers include bicomponent fibers, e.g., core-sheath configuration fibers with a sheath component of relatively lower melting temperature at which fusion bonding will occur, the resilient polymer fibers may comprise the constituent chemistry of only the core, which may be a polymer having a relatively higher melting temperature.

Nonwoven performance can be impacted by a combination of the nonwoven fiber polymer choice, fiber properties, and how the fibers are arranged or connected. Nonwoven selection can impact the absorbent article's ability to recover its shape following compression, bending and extension (stretching) forces present in-use with body motion. If the fibers are short (less than about 10 mm), the fibers are likely to irreversibly rearrange under extension and compressive forces. The rearranging (changing their orientation/state) of fibers in a fiber matrix dissipates the tensile (elongation) or compressive forces so that the energy used to affect the deformation is no longer available for recovery to the original shape. Longer fiber networks (typically greater than about 10 mm but less than about 100 mm) can absorb the tensile/compressive forces typical of bodily motions along the fiber length and across the structure. As a result, the absorbed forces are available to recover the structure to its original state. Longer fiber networks composed of finer fibers (typically less than about 15 to about 20 microns) more readily elongate and compress. As a result, the fluff/AGM structure can deform more readily (and to a higher degree) but the energy associated with these deformations is relatively small and insufficient to carry the structure back to its original state. Thicker fiber, such as greater than about 20 microns or about 2.0 DTex to about 10 DTex, are both flexible under bodily forces but provide sufficient fiber and web recovery energy to return the structure to its original state.

The fiber arrangement in a long fiber network from a structural standpoint can impact the performance of the absorbent articles containing these nonwovens. Long fiber webs of thicker fibers are typically loftier than a conventional thin spunbond nonwoven web composed of continuous fine fibers that are closely spaced and physically bonded together. Creating a web of thicker fibers arranged in a more randomized orientation such as those that can be achieved via carding, hydro-entangling, and needling are able to elongate and compress, whereby the fibers only temporary adjust their arrangement (space between the fibers exist for these arrangements) and are able to carry/store the deformation forces and this energy is available for recovering the structural shape.

Additionally, finer (less than about 2.0 DTex) synthetic fibers such as BiCo and PP fibers commonly found in spunbond are closely spaced, relatively parallel aligned and closely bonded together. The bonded fibers within these spunbond webs are so interconnected (with closely spaced point bonds) that in tensile (elongation) the fibers at the polymer level are forced to stretch, resulting in polymer chains within the fiber permanently rearranging. As a result, the fibers themselves potentially remain permanently elongated (permanently strained) and are no longer able to recover to their initial state.

In some configurations, the polymer fibers in the upper nonwoven layer and the polymer fibers of the lower nonwoven layer may be different. In some configurations, the polymer fibers of the upper nonwoven layer and the polymer fibers of the lower nonwoven layer may be the same. In some configurations, the upper nonwoven layer may be carded nonwoven. In some configurations, the upper nonwoven layer may be air through bonded or hydroentangled. In some configurations, the upper nonwoven layer is not a spunbond material.

Suitable nonwoven materials examples may include, but are not limited to, the following materials: (i) a 40 gsm carded resilient nonwoven material produced by Yanjan China (material code; ATB Z87G-40-90) which is a carded nonwoven composed of a blend of 60% 2 DTex and 40% 4 DTex BiCo (PE/PET) fibers. The fibers are bonded (ATB=Through ‘hot’ Air Bonded) to create a wet resilient network. Without being limited by theory, it is believed that because of the presence of the 4 DTex BiCo fibers and the fiber-to-fiber bonded BiCo network, the material has a low Permanent Strain (less than about 0.013 mm/mm) and a sufficient Dry Recovery Energy (greater than about 0.03 N*mm) in the Dry CD Ultra Sensitive 3 Point Bending Method; (ii) a 55 gsm resilient spunlace material produced by Sandler Germany (material code: 53FC041001), which is a hydro-entangled nonwoven that is produced via a carding step (like the nonwoven described above) followed by hydro-entangling with an elevated drying step (as described in U.S. Patent Publication No. 2020/0315873A1) that creates both an entangled and BiCo bonded resilient network. It comprises a fiber blend of 30% 10 DTex HS-PET, 50% 2.2 DTex BiCo (PE/PET), and 20% 1.3 DTex rayon. The material has a low Permanent Strain (less than about 0.013 mm/mm) and a sufficient Dry Recovery Energy (greater than about 0.03 N*mm) in the Dry CD Ultra Sensitive 3 Point Bending Method; and (iii) a 50 gsm resilient spunlace material produced by Sandler Germany (material code: 53FC041005 opt82), which is a hydro-entangled nonwoven that is produced via a carding step (like the nonwoven described above) followed by hydro-entangling with an elevated drying step (as described in U.S. Patent Publication No. 2020/0315873A1) that creates both an entangled and BiCo bonded resilient network. It comprises a fiber blend of 60% 5.8 DTex BiCo (PE/PET), 20% 3.3 DTex tri-lobal ‘structural’ rayon, and 20% 1.3 DTex rayon. The material has a low Permanent Strain (less than about 0.013 mm/mm) and a sufficient Dry Recovery Energy (greater than about 0.03 N*mm) in the Dry CD Ultra Sensitive 3 Point Bending Method. While this material has 40% rayon that can soften when wet, the use of structural tri-lobal rayon fibers helps structural stability in the wet state.

In combination with adjustment of pore size, volume, and number via selection of appropriate fiber size, basis weight, and extent of consolidation, the manufacturer may wish to select fiber constituents for having particular surface chemistry(ies), e.g., fibers with hydrophobic surfaces, hydrophilic surfaces, or a blend of differing fibers and/or z-direction stratification or gradient thereof. Fibers having hydrophilic surfaces will tend to attract and move aqueous components of menstrual fluid there along in a manner conducive to wicking and rapid fluid acquisition following discharge. At the same time, however, a predominance of hydrophilic fibers surfaces within the topsheet may increase a tendency of the topsheet to reacquire fluid from absorbent components beneath (rewet), which can cause an undesirable wet feel for the user. On the other hand, fibers having hydrophobic surfaces will tend to repel aqueous components of menstrual fluid and/or resist movement of fluid along their surfaces, thereby tending to resist wicking—but also to resist rewetting. The manufacturer may wish to seek an appropriate balance in selecting constituent fibers having hydrophilic surfaces, fibers having hydrophobic surfaces, or a blend and/or z-direction stratification thereof, in combination with fiber size, fiber consolidation level, and resulting topsheet pore size, volume and number, for any particular product design.

The inner core layer is produced in an airlaying process. Streams of cellulose and superabsorbent polymer are carried on a fast moving airstream and deposited into a three dimensionally shaped pocket on a rotating forming drum with a vacuum below to draw the cellulose and superabsorbent polymer into the pocket in a laydown station. This shaped pocket provides the actual physical shape of the absorbent core structure. The upper nonwoven layer may be first introduced onto the forming drum and under the vacuum the upper nonwoven layer is drawn into the 3-dimensional pocket shape. In this case, the cellulose and superabsorbent polymer material stream is deposited on the upper nonwoven layer directly in the forming station. Prior to entering the forming station, the nonwoven is coated with an adhesive to provide a stronger connection of the cellulose and superabsorbent polymer to the nonwoven layer. On exiting the laydown section, the lower nonwoven layer is combined with the upper nonwoven layer carrying the cellulose and superabsorbent polymer layer exiting the laydown section. This lower nonwoven is precoated with adhesive to enable a perimeter seal and to better integrate the cellulose and superabsorbent polymer without hindering the flow of liquid into the cellulose and superabsorbent polymer matrix. These adhesives are not represented in the Figures for simplicity.

The inner core layer may comprise any of a wide variety of liquid-absorbent materials commonly used in absorbent articles, such as comminuted wood pulp, which is generally referred to as airfelt. One suitable absorbent core material is an airfelt material which is available from Weyerhaeuser Company, Washington, USA, under Code No. FR516. Examples of other suitable liquid-absorbent materials for use in the absorbent core may include creped cellulose wadding; meltblown polymers including coform; chemically stiffened, modified or cross-linked cellulosic fibers; synthetic fibers such as crimped polyester fibers; peat moss; cotton, bamboo; absorbent polymer materials; or any equivalent material or combinations of materials, or mixtures of these.

Absorbent polymer materials for use in absorbent articles typically comprise water-insoluble, water-swellable, hydrogel-forming crosslinked absorbent polymers which are capable of absorbing large quantities of liquids and of retaining such absorbed liquids under moderate pressure.

The absorbent polymer material for the absorbent cores according to the present disclosure may comprise superabsorbent particles, also known as “superabsorbent materials” or as “absorbent gelling materials”. Absorbent polymer materials, typically in particle form, may be selected among polyacrylates and polyacrylate based materials, such as for example partially neutralized, crosslinked polyacrylates. The term “particles” refers to granules, fibers, flakes, spheres, powders, platelets and other shapes and forms known to persons skilled in the art of superabsorbent particles. In some aspects, the superabsorbent particles may be in the shape of fibers, i.e., elongated, acicular superabsorbent particles.

In some configurations, the inner core layer may comprise cellulosic fibers and superabsorbent particles. The inner core layer may comprise from about 50% to about 85% cellulosic fibers, or from about 55% to about 80%, or from about 60% to about 75%, all by weight of the inner core layer. The inner core layer may comprise from about 10% to about 50% superabsorbent particles, or from about 15% to about 50%, or from about 20% to about 40%, or from about 25% to about 35%, all by weight of the inner core layer. In some configurations, the inner core layer may comprise from about 125 gsm to about 500 gsm cellulosic fibers. In some configurations, the inner core layer may comprise from about 125 gsm to about 300 gsm superabsorbent particles.

In some configurations, the inner core layer may comprise from about 50% to about 85% cellulosic fibers and from about 15% to about 50% superabsorbent particles.

The absorbent article 20 may be resilient and conformable and may deliver a superior in-use experience without substantially bunching and/or compressing. The absorbent article may be exposed to bodily forces and may recover to its original state.

### Topsheet

Topsheet 110 may be formed of any suitable nonwoven web or formed film material. Referring back to the figures, the topsheet 110 is positioned adjacent a wearer-facing surface of the absorbent article 20 and may be joined thereto and to the backsheet 130 by any suitable attachment or bonding method. The topsheet 110 and the backsheet 130 may be joined directly to each other in the peripheral regions outside the perimeter of the absorbent core structure and may be indirectly joined by directly joining them respectively to wearer-facing and outward-facing surfaces of the absorbent article or additional optional layers included with the absorbent article.

The absorbent article 20 may have any known or otherwise effective topsheet 110, such as one which is compliant, soft feeling, and non-irritating to the wearer's skin. A suitable topsheet material will include a liquid pervious material that is comfortable when in contact with the wearer's skin and permits discharged menstrual fluid to rapidly penetrate through it. Some suitable examples of topsheet materials include films, nonwovens, laminate structures including film/nonwoven layers, film/film layers, and nonwoven/nonwoven layers.

Nonlimiting examples of nonwoven web materials that may be suitable for use to form the topsheet 110 include fibrous materials made from natural fibers, modified natural fibers, synthetic fibers, or combinations thereof. Some suitable examples are described in U.S. Pat. Nos. 4,950,264; 4,988,344; 4,988,345; 3,978,185; 7,785,690; 7,838,099; 5,792,404; and 5,665,452.

The topsheet 110 may be compliant, soft feeling, and non-irritating to the wearer's skin. Further, the topsheet 110 may be liquid pervious permitting liquids (e.g., urine, menses) to readily penetrate through its thickness. Some suitable examples of topsheet materials include films, nonwovens, laminate structures including film/nonwoven layers, film/film layers, and nonwoven/nonwoven layers. Other exemplary topsheet materials and designs are disclosed in U.S. Patent Application Publication Nos. 2016/0129661, 2016/0167334, and 2016/0278986.

In some examples, the topsheet 110 may include tufts as described in U.S. Pat. Nos. 8,728,049; 7,553,532; 7,172,801; 8,440,286; 7,648,752; and 7,410,683. The topsheet 20 may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535; and US Patent Publication No. 2015/041640. Another suitable topsheet may be formed from a three-dimensional substrate as detailed in US 2017/0258647. The topsheet may have one or more layers, as described in US Patent Publication Nos. 2016/0167334; US 2016/0166443; and US 2017/0258651.

In some examples a topsheet 110 may be formed of a nonwoven web material of a spunbond web including single-component continuous fibers, or alternatively, bi-component or multi-component fibers, or a blend of single-component fibers spun of differing polymer resins, or any combination thereof. The topsheet may also be a formed nonwoven topsheet as disclosed in US Patent Publication No. 2019/0380887.

In order to ensure that fluid contacting the top (wearer-facing) surface of a topsheet will move suitably rapidly in a z-direction to the bottom (outward-facing) surface of the topsheet where it can be drawn into the absorbent article, it may be important to ensure that the nonwoven web material forming the topsheet has an appropriate weight/volume density, reflecting suitable presence of interstitial passageways (sometimes known as “pores”) among and between the constituent fibers, through which fluid may move within the nonwoven material. In some circumstances a nonwoven material with fibers that are consolidated too densely may have insufficient numbers and/or volumes and/or sizes of pores, and the nonwoven will obstruct rather than facilitate rapid downward z-direction fluid movement. On the other hand, a nonwoven with fibers that are too large and/or not consolidated enough to provide a certain level of opacity (for purposes of concealing absorbed fluid in the layers beneath) and a substantial appearance may be negatively perceived by users.

The caliper of the topsheet material may be controlled, to balance competing needs for opacity and loft (which call for a higher caliper) vs. a limitation on the z-direction distance that discharged fluid travels through the topsheet from the wearer-facing surface to the outward-facing surface to reach the absorbent core structure below. Thus, it may be desired that the manufacture of the topsheet material be controlled to produce a topsheet material having a caliper of from about 0.20 mm to about 1.0 mm, or from about 0.25 mm to about 0.80 mm, or from about 0.30 mm to about 0.60 mm.

### Secondary Topsheet (STS)

An STS layer may be included, in some circumstances, between the topsheet and the absorbent core structure to enable the absorbent core structure to readily receive a sudden discharge of fluid, and after receipt, to wick it along x- and y-directions to distribute it across the underlying absorbent core structure.

If included, an STS may be a nonwoven fibrous structure which may include cellulosic fibers, non-cellulosic fibers (e.g., fibers spun from polymer resin(s)), or a blend thereof. To accommodate the folding and lateral gathering of the absorbent article 20, and of the absorbent core structure 10, as described herein, the STS may be formed of a material that is relatively pliable (i.e., has relatively low bending stiffness).

A number of particular examples of suitable STS compositions and structures, as well as combinations thereof with suitable topsheet compositions and structures, are further described in U.S. applications. Ser. Nos. 16/831,862; 16/831,854; 16/832,270; 16/831,865; 16/831,868; 16/831,870; and Ser. No. 16/831,879; and U.S. Provisional Apps. Ser. Nos. 63/086,610 and 63/086,701. Additional suitable examples are described in U.S. Pat. No. 9,504,613; WO 2012/040315; and US 2019/0021917.

In some configurations, the absorbent article may be free of a secondary topsheet.

### Backsheet

The backsheet 130 may be positioned beneath or subjacent an outward-facing surface of the absorbent core structure 10 and may be joined thereto by any suitable attachment methods. For example, the backsheet 130 may be secured to the absorbent core structure 10 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent core structure 10 is not joined directly to the backsheet 130.

The backsheet 130 may be impermeable or substantially impermeable by aqueous liquids (e.g., urine, menstrual fluid) and may be manufactured from a thin plastic film, although other flexible liquid impermeable materials may also be used. As used herein, the term “flexible” refers to materials which are compliant and will readily conform to the general shape and contours of the human body. The backsheet 130 may prevent, or at least substantially inhibit, fluids absorbed and contained within the absorbent core structure 10 from escaping and reaching articles of the wearer's clothing which may contact the absorbent article 20, such as underpants and outer clothing. However, in some instances, the backsheet 130 may be made and/or adapted to permit vapor to escape from the absorbent core structure 10 (i.e., the backsheet is made to be breathable), while in other instances the backsheet 130 may be made so as not to permit vapors to escape (i.e., it is made to be non-breathable). Thus, the backsheet 130 may comprise a polymeric film such as thermoplastic films of polyethylene or polypropylene. A suitable material for the backsheet 130 is a thermoplastic film having a thickness of from about 0.012 mm (0.5 mil) to about 0.051 mm (2.0 mils), for example. Any suitable backsheet known in the art may be utilized with the present invention.

Some suitable examples of materials suitable for forming a backsheet are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523; 3,989,867; 3,156,242; WO 97/24097; U.S. Pat. Nos. 6,623,464; 6,664,439; and 6,436,508.

The backsheet 130 may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489; 4,341,216; 4,713,068; 4,818,600; EP 203 821; EP 710 471; EP 710 472; and EP 0 793 952.

### Other Features

In some configurations, the absorbent article 20 may comprise a panty fastening component, such as a panty fastening adhesive or components of a hook and-loop fastening system (such as VELCRO®).

In some configurations, the absorbent article 20 may be provided with a panty fastening adhesive disposed on the garment-facing side of the backsheet 130 in order to provide a mechanism for the user to adhere the absorbent article to the inside of her underpants in the crotch region thereof. The panty fastening adhesive may comprise any adhesive or glue used in the art for such purposes. These adhesives typically are pressure sensitive and remain tacky well below their application temperature. In some configurations, the panty fastening adhesive may be a pressure sensitive hot melt adhesive. When the absorbent article 20 is packaged for shipping, handling and storage prior to use, the panty fastening adhesive may be covered by one or more sheets of release film or paper that covers/shields the adhesive deposits from contact with other surfaces until the user is ready to remove the release film or paper and place the absorbent article in her underpants for wear/use. The release film or paper may also function as an individualized packaging for the article or provide a disposal function as known in the art. Any commercially available release paper or film may be used. Suitable examples include BL 30 MG-A SILOX EI/O, BL 30 MG-A SILOX 4 P/O available from Akrosil Corporation, and M&W films available from Gronau in Germany, under the code X-5432. In some configurations, the absorbent article may be packaged in a bi-folded or tri-folded state.

In some configurations, the absorbent article 20 may include opposing wing portions 140, 150 on each side, extending laterally outward from a first longitudinal side 141 and a second longitudinal side 151 of the absorbent article. Wings are currently commonly provided with feminine hygiene absorbent articles. As provided, they typically have deposits of adhesive applied to their outward-facing surfaces (surface are outward-facing prior to placement of the absorbent article within the user's underwear and application of the wings). The wing portions may also include deposits of adhesive as described above, which enable the user to wrap the wing portions through the leg openings of the underpants and around the inside edges thereof, and adhere the wing portions to the outward-facing surface/underside of the underpants in the crotch region, providing supplemental holding support for the absorbent article and helping guard the underpants proximate the leg edges thereof against soiling.

### Test Methods

### Layers of Interest

For any of the methods below in which all the component layers of an article will not be tested, the layers of interest may be separated using cryo-spray as needed from layers which will not be tested.

### Strain to Break Method

The force versus displacement behavior of a sample is measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The sample is subjected to tensile elongation at a constant rate (mm/sec) until it breaks, and the percent strain to break is measured. All testing is performed in a room controlled at 23° C.±3° C. and 50%±2% relative humidity and test samples are conditioned in this environment for at least 2 hours prior to testing.

The fixtures used to grip the test specimen are lightweight (<80 grams), vise action clamps with half cylinder steel versus rubber coated steel grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is prepared as follows. The test specimen is cut from an area on the test material that is free of any folds or wrinkles. The test specimen is 100 mm long (parallel to the lateral axis, or intended lateral axis of the article) and 25.4 mm wide (parallel to the longitudinal axis, or intended longitudinal axis of the article). In like fashion, five replicate test specimens are prepared.

Prepare the universal test frame as follows. Set the initial grip to grip separation distance to a nominal gage length of 80 mm, then zero the crosshead. Program the test frame to move the grips closer together by an intentional slack of 1 mm to ensure no pretension force exists on the test specimen at the onset of the test. (During this motion, the specimen will become slack between the grips.) Next, the grips will move apart at a slack speed of 1 mm/s until the slack preload of 0.05 N is exceeded. (At this point, the crosshead position signal is used to compute the sample slack, the adjusted gage length, and the strain is defined at zero, 0.0). The grips will then move apart at a speed of 1 mm/s until the sample breaks or the extension limit of the instrument is exceeded.

The test is executed by inserting the test specimen into the grips such that the long axis of the specimen is parallel and centered with the motion of the crosshead. Start the test and continuously collect force (“load”) and displacement data at a data acquisition rate of 100 Hz.

Construct a graph of load (N) versus displacement (mm). Determine the peak load from the curve, then determine the break sensitivity as follows. Determine the crosshead position at which the load signal decreases by 75% after the peak load is reached, and record as specimen final length (Lf) to the nearest 0.01 mm. The initial length of the specimen is defined by the crosshead position when the slack preload of 0.05 N is exceeded, and this value is recorded as specimen initial length (Li) to the nearest 0.01 mm. Calculate the percent strain to break as follows, and record to the nearest 1 percent.

% Strain to Break=((Lf−Li)/Li)*100

In like fashion, the procedure is repeated for all five replicate test specimens. The arithmetic mean of % strain to break among the five replicate test specimens is calculated and reported as % Strain to Break to the nearest 1 percent.

### Dry MD 3 Point Bend Method

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry test specimens. The intention of this method is to mimic deformation created in the x-y plane by a wearer of an absorbent article during normal use. All testing is performed in a room controlled at 23° C.±3° C. and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be moved horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place, the bar of the top fixture is parallel to and aligned front to back with the bars of the bottom fixture and is centered between the bars of the bottom fixture. Both fixtures include an integral adapter appropriate to fit the respective position on the universal test frame and lock into position such that the bars are orthogonal to the motion of the crossbeam of the test frame.

Set the gap (“Span”) between the bars of the lower fixture to 25 mm±0.5 mm (center of bar to center of bar) with the upper bar centered at the midpoint between the lower bars. Set the gage (bottom of top bar to top of lower bars) to 1.0 cm.

The thickness (“caliper”) of the test specimen is measured using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as caliper to the nearest 0.01 mm.

The absorbent article samples are conditioned at 23° C.±3° C. and 50%±2% relative humidity two hours prior to testing. Remove the test sample from its outer wrapper, then remove the protective cover/release paper from the panty fastening adhesive on the garment facing side of the sample. Lightly apply talc powder to the adhesive to mitigate any tackiness. For each test sample, dry test specimens are prepared from two separate test locations on the sample from areas that are free from any folds or wrinkles, as follows. Referring to FIG. 2, the first test specimen is obtained from a location on the test sample within the middle region 22 such that the center of the test specimen is the intersection of the lateral midpoint of the absorbent article and line 90. The second test specimen is obtained from the rear-most position possible (inbound of the perimeter seal 230) on the test sample within the rear region 23 such that the center of the test specimen is the lateral midpoint of the absorbent article. The dry specimens are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, marking the body-facing surface (or the surface intended to face the body of a finished article), and labeling as “middle region” or “rear region” to correspond to each of the test locations. Measure the thickness of each of the test specimens, as described herein, and record as dry specimen caliper to the nearest 0.01 mm, denoting middle or rear region for each. In like fashion, five replicate dry test specimens are prepared from the middle and rear test locations on five separate test samples.

Program the universal test frame for a flexural bend test, to move the crosshead such that the top fixture moves down with respect to the lower fixture at a rate of 1.0 mm/sec until the upper bar touches the top surface of the specimen with a nominal force of 0.02 N, then continue for an additional 12 mm. The crosshead is then immediately returned to the original gage at a rate of 1.0 mm/s. Force (N) and displacement (mm) data are continuously collected at 100 Hz throughout the test.

Load a dry test specimen such that it spans the two lower bars and is centered under the upper bar, with its sides parallel to the bars. For MD bending, the MD direction of the test specimen is perpendicular to the length of the 3 bars. Start the test and continuously collect force and displacement data.

Construct a graph of force (N) versus displacement (mm). From the graph, determine the maximum peak force and record as dry MD peak load to the nearest 0.01 N, denoting the test location as middle or rear region. Now calculate the maximum slope of the curve between initial force and maximum force (during the loading portion of the curve) and record to the nearest 0.1 unit. Calculate the modulus as follows, and record as dry MD modulus to the nearest 0.001 N/mm², denoting the test location as middle or rear region.

$$MD Dry Bending Modulus ( N / {mm}^{2} ) = ( Slope \times {( Spam}^{3} ) ) / ( 4 \times specimen width \times ( specimen {caliper}^{3} ) )$$

Calculate bending stiffness as follows, and record as dry MD bending stiffness to the nearest 0.1 N mm², denoting the test location as middle or rear region.

In like fashion, the procedure is repeated for all five replicates of the dry test specimens from the middle region and all five replicates of the dry test specimens from the rear region. The arithmetic mean among the five replicate dry test specimens representing the middle and rear regions is calculated separately for each of the parameters and reported as Dry MD Peak Load to the nearest 0.01 N, Dry MD Bending Modulus to the nearest 0.001 N/mm², and Dry Bending Stiffness to the nearest N mm², with each parameter labeled appropriately to denote middle or rear test locations.

### Dry CD Ultra Sensitive 3 Point Bending Method

The CD (cross-direction) bending properties of a test sample are measured using an ultra sensitive 3 point bend test on a universal constant rate of extension test frame (a suitable instrument is the MTS Alliance using TestSuite Software, as available from MTS Systems Corp., Eden Prairie, MN, or equivalent) equipped with a load cell appropriate for the forces being measured. The intention of this method is to mimic deformation created in the x-y plane by a wearer of an absorbent article during normal use. All testing is performed in a room controlled at 23° C.±3° C. and 50%±2% relative humidity and test samples are conditioned in this environment for at least 2 hours prior to testing.

The ultra sensitive 3 point bend method is designed to maximize the force signal to noise ratio when testing materials with very low bending forces. The force signal is maximized by using a high sensitivity load cell (e.g., 5 N), using a small span (load is proportional to the span cubed) and using a wide specimen width (total measured load is directly proportional to width). The fixture is designed such that the bending measurement is performed in tension, allowing the fixture mass to be kept to a minimum. Noise in the force signal is minimized by holding the load cell stationary to reduce mechanical vibration and inertial effect and by making the mass of the fixture attached to the load cell as low as possible.

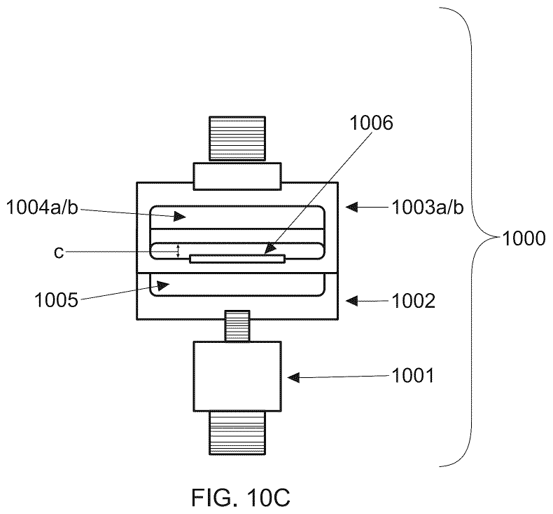

Referring to FIGS. 10A-10C, the load cell 1001 is mounted on the stationary crosshead of the universal test frame. The ultra sensitive fixture 1000 consists of three thin blades constructed of a lightweight, rigid material (such as aluminum, or equivalent). Each blade has a thickness of 1.0 mm, rounded edges and a length that is able to accommodate a bending width of 100 mm. Each of the blades has a cavity 1004a and 1004b (outside blades) and 1005 (central blade) cut out to create a height, h, of 5 mm of blade material along their horizontal edges. The two outside blades 1003a and 1003b are mounted horizontally to the moveable crosshead of the universal test frame, aligned parallel to each other, with their horizontal edges vertically aligned. The span, s, between the two outside blades 1003a and 1003b is 5 mm±0.1 mm (inside edge to inside edge). The central blade 1002 is mounted to the load cell on the stationary crosshead of the universal test frame. When in place, the central blade 1002 is parallel to the two outside blades 1003a and 1003b and centered at the midpoint between the outside blades 1003a and 1003b. The blade fixtures include integral adapters appropriate to fit the respective positions on the universal test frame and lock into position such that the horizontal edges of the blades are orthogonal to the motion of the crossbeam of the universal test frame.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained as follows. The test specimen is cut from an area on the test material that is free of any folds or wrinkles. The dry specimens are prepared for CD bending (i.e., bending normal to the lateral axis of the sample) by cutting them to a width of 50.0 mm along the CD (cross direction; parallel to the lateral axis of the sample) and a length of 100.0 mm along the MD (machine direction; parallel to the longitudinal axis of the sample), maintaining their orientation after they are cut and marking the body-facing surface (or the surface intended to face the body of a finished article). In like fashion, five replicate dry test specimens are prepared.

The universal test frame is programmed such that the moveable crosshead is set to move in a direction opposite of the stationary crosshead at a rate of 1.0 mm/s. Crosshead movement begins with the specimen 1006 lying flat and undeflected on the outer blades 1003a and 1003b, continues with the inner horizontal edge of cavity 1005 in the central blade 1002 coming into contact with the top surface of the specimen 1006, and further continues for an additional 4 mm of crosshead movement. The crosshead stops at 4 mm and then immediately returns to zero at a speed of 1.0 mm/s. Force (N) and displacement (mm) are collected at 50 Hz throughout.

Prior to loading the test specimen 1006, the outside blades 1003a and 1003b are moved towards and then past central blade 1002 until there is approximately a 3 mm clearance, C, between the inner horizontal edges of cavities 1004a and 1004b in the outside blades 1003a and 1003b and the inner horizontal edge of cavity 1005 in the central blade 1002 (see FIG. 10C). The specimen 1006 is placed within clearance C such that it spans the inner horizontal edges of cavities 1004a and 1004b in the outside blades 1003a and 1003b, oriented such that the MD (short side) of the specimen is perpendicular to the horizontal edges of the blades and the body-facing surface of the specimen is facing up. Center the specimen 1006 between the outside blades 1003a and 1003b. Slowly move the outside blades 1003a and 1003b in a direction opposite of the stationary crosshead until the inner horizontal edge of cavity 1005 in the central blade 1002 touches the top surface of the specimen 1006. Start the test and continuously collect force and displacement data.

Force (N) is plotted versus displacement (mm). The maximum peak force is recorded to the nearest 0.001 N. The area under the curve from load onset up to the maximum peak force is calculated and recorded as bending energy to the nearest 0.001 N*mm. The recovery energy is calculated as the area under the curve where the force is unloaded from the maximum peak to 0.0 N and recorded as recovery energy to the nearest 0.001 N*mm. In like fashion, repeat the entire test sequence for a total of five dry test specimens and five wet test specimens.

For each test specimen, the arithmetic mean of the maximum peak force among like specimens is calculated to the nearest 0.001 N and recorded as Dry Peak Load. For each test specimen, the arithmetic mean of bending energy among like specimens is calculated to the nearest 0.001 N*mm and reported as Dry Bending Energy. For each test specimen, the arithmetic mean of recovery energy among like specimens is calculated to the nearest 0.001 N*mm and reported as Dry Recovery Energy.

### CD Cyclic Elongation to 3% Strain Method

The cyclic tensile and recovery response of absorbent article specimens are measured for ten cycles of load application (“elongation”) and load removal (“recovery”) using a universal constant rate of extension test frame. The test specimen is cycled ten times to 3% engineering strain, then back to zero engineering strain. For each cycle, stiffness, peak load, normalized energy to peak, normalized recovery energy, strain at start of cycle, and strain at end of cycle (i.e., “permanent strain”) are calculated and reported. The intention of this method is to understand the ability of samples to stretch in the x-y plane as a result of bodily forces, and then recover to their original state. All measurements are performed in a laboratory maintained at 23° C.±2° C. and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is cut from an area on the test material that is free of any residual of folds or wrinkles. The test specimen is as long as the lateral length of the article (parallel to the lateral axis of the article, or the intended lateral axis of the article). When excising specimens from absorbent articles of different sizes and widths, the total specimen length (Lₜₒₜₐₗ) may vary from product to product, thus the results will be normalized to compensate for this variation. The test specimen has a width of 25.4 mm wide (parallel to the longitudinal axis, or intended longitudinal axis of the article). Specimen width (w)=25.4 mm. Measure and record the total specimen length (Lₜₒₜₐₗ) to the nearest 0.1 mm. In like fashion, five replicate test specimens are prepared.

Measure the thickness (t) of the test specimen using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as specimen thickness (t) to the nearest 0.01 mm.

Prepare the universal test frame as follows. Set the initial grip to grip separation distance to a nominal gage length (Lₙₒₘᵢₙₐₗ) that is shorter than the total specimen length and such that the specimen can be gripped securely at both ends (i.e., Lₙₒₘᵢₙₐₗ<Lₜₒₜₐₗ). Then zero the crosshead. Program the test frame to move the grips closer together by an intentional slack of 1 mm to ensure no pretension force exists on the test specimen at the onset of the test. (During this motion, the specimen will become slack between the tensile grips.) Next, the grips will move apart at a slack speed of 1 mm/s until the slack preload of 0.05 N is exceeded. At this point, the following are true. 1) The crosshead position signal (mm) is defined as the specimen slack (L_{slack}). 2) The initial specimen gage length (L₀) is calculated as the nominal gage length plus the slack L₀=Lₙₒₘᵢₙₐᵢₗ+L_{slack}, where units are in millimeters. 3) The crosshead extension (ΔL) is set to zero (0.0 mm). 4) The crosshead displacement (mm) is set to zero (0.0 mm). At this position the engineering strain is zero, 0.0. Engineering strain is calculated as the change in length (ΔL) divided by the initial length (L₀). Engineering strain=ΔL/L₀. For one test cycle, the grips move apart at the initial speed of 1 mm/s until the engineering strain endpoint of 0.03 mm/mm is exceeded, immediately followed by the grips moving toward each other at the initial speed of 1 mm/s until the crosshead signal becomes less than the crosshead return position of 0 mm. The test cycle is repeated until a total of 10 cycles is complete.

The test is executed by inserting the test specimen into the grips such that the long axis of the specimen is parallel and centered with the motion of the crosshead. Start the test and continuously collect time, force and displacement data at a data acquisition rate of 100 Hz.

Construct a graph of load (N) versus displacement for all ten cycles. For each cycle, perform the following. Record peak load to the nearest 0.01 N. Calculate the energy to peak (Eₚₑₐₖ) as the area under the load versus displacement curve from the cycle start to the strain endpoint of 0.03 mm/mm (during the loading portion of the cycle) and record to the nearest 0.01 N*mm. Calculate the return energy (Eᵣₑₜᵤᵣₙ) as the area under the load versus displacement curve from the strain endpoint of 0.03 mm/mm to the crosshead return of 0 mm (during the unloading portion of the cycle) and record as recovery energy to the nearest 0.01 N*mm. Calculate the normalized energy to peak (NEₚₑₐₖ) as the energy to peak divided by the initial length, where NEₚₑₐₖ=Eₚₑₐₖ/L₀, and record to the nearest 0.01 mN. Calculate the normalized return energy (NEᵣₑₜᵤᵣₙ) as the return energy divided by the initial length (NEᵣₑₜᵤᵣₙ=Eᵣₑₜᵤᵣₙ/L₀), and record to the nearest 0.01 mN. Units of NEₚₑₐₖ and NEᵣₑₜᵤᵣₙ are milliNewtons (mN).

Now construct a graph of engineering stress (a) versus engineering strain for all ten cycles, and for each cycle perform the following. Engineering stress, in units of N/mm², is the load divided by the cross sectional area of the specimen, where the cross sectional area is the specimen width (w) multiplied by the thickness (t), σ=Load/(w*t). Determine the modulus, or slope of the stress versus strain curve for a line between the point that occurs at the minimum force and the point that occurs at the maximum force (during the loading portion of the cycle) and record as modulus to the nearest 0.01 N/mm². Calculate stiffness by multiplying the modulus by the specimen thickness and record as tensile stiffness to the nearest 0.01 N/mm. The strain of the test specimen at the beginning of the cycle is defined by the strain when the slack preload of 0.05 N is exceeded for that cycle (during the loading portion of the cycle), and is recorded as cycle initial strain to the nearest 0.01 mm/mm. The strain of the test specimen at the end of the cycle is defined by the strain when the load becomes less than the preload of 0.05 N for that cycle (during the unloading portion of the cycle), and is recorded as permanent strain to the nearest 0.01 mm/mm. In like fashion, the overall procedure is now repeated for all five replicates.

The arithmetic mean among the five replicate test specimens is calculated for each of the parameters, for each of the ten cycles, and reported as Peak Load to the nearest 0.01 N, Normalized Energy to Peak to the nearest 0.01 mN, Normalized Recovery Energy to the nearest 0.01 mN, Tensile Stiffness to the nearest 0.01 N/mm, Cycle Initial Strain to the nearest 0.01 mm/mm, and Permanent Strain to the nearest 0.001 mm/mm.

### Structural Bond Sites Pattern Spacing and Area Measurement Method

The spacing between the discreet structural bond sites that are used to create a quilt-like pattern on absorbent article samples, and the overall area taken up by the sum of those elements in a specified region of the sample are measured on images of the absorbent article sample acquired using a flatbed scanner. The scanner is capable of scanning in reflectance mode at a resolution of 2400 dpi and 8 bit grayscale. A suitable scanner is an Epson Perfection V750 Pro from Epson America Inc., Long Beach CA, or equivalent. The scanner is interfaced with a computer running an image analysis program. A suitable program is ImageJ v. 1.52, National Institute of Health, USA, or equivalent. The sample images are distance calibrated against an acquired image of a ruler certified by NIST. To enable maximum contrast, the specimen is backed with an opaque, black background of uniform color prior to acquiring the image. All testing is performed in a conditioned room maintained at about 23±2° C. and about 50±2% relative humidity.

The test sample is prepared as follows. Remove the absorbent article from any wrapper present. If the article is folded, gently unfold it and smooth out any wrinkles. If wings are present, extend them but leave the release paper intact. The test samples are conditioned at about 23° C.±2° C. and about 50%±2% relative humidity for 2 hours prior to testing.

Images are obtained as follows. The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and the test sample is scanned under the same scanning conditions as follows. Place the test sample onto the center of the scanner glass and secure, if necessary, such that it lies flat with the body-facing surface of the sample facing the scanner's glass surface. The sample is oriented in such a way that the entire sample is within the glass surface. The black background is placed on top of the specimen, the scanner lid is closed, and a scanned image of the entire sample is acquired with the same settings as used for the calibration image. The sample image is saved as an uncompressed TIFF format file.

The sample image is analyzed as follows. Open the calibration image file in the image analysis program, and calibrate the image resolution using the imaged ruler to determine the number of pixels per millimeter. Now open the sample image in the image analysis program, and set the distance scale using the image resolution determined from the calibration image. Now visually inspect the pattern of emboss elements present on the sample in the image and identify the zones of the pattern that are to be analyzed. For example the absorbent article can be divided into three equal lengths zones in the machine direction such as the front one third zone, zone 1, the central one third zone, zone 2 and the end one third zone, zone 3 as example. Use the image analysis tools to draw a shape along the outer perimeter of the first discreet zone to be analyzed. Measure the area of this first zone and record as Zone 1 Total Area to the nearest 0.01 mm². Now measure the area of each individual, discreet emboss element that lies inside of the zone 1 perimeter as follows. Draw a minimum bounding circle around an individual emboss element such that no portion of the emboss element lies outside of the bounding circle. Now measure the area of the bounding circle for that emboss element and record the emboss element area to the nearest 0.01 mm². In like fashion, measure the area of every emboss element, including portions of emboss elements, that lie inside zone 1 and record each to the nearest 0.01 mm². Now sum the areas of all of the emboss elements inside of zone 1 and record as Zone 1 Total Emboss Element Area to the nearest 0.01 mm². Divide the Zone 1 Total Emboss Element Area by the Zone 1 Total Area then multiply by 100 and record as Zone 1% Total Area Represented by Emboss Elements. The spacing between each discreet emboss element inside of zone 1 is measured as follows. Measure the distance from the center of the minimum bounding circle drawn around a discreet emboss element inside of zone 1, as described herein, to the center of the minimum bounding circle drawn around the nearest neighboring discreet emboss element inside of zone 1, and record this distance as emboss spacing to the nearest 0.01 mm. In like fashion, repeat for all neighboring emboss elements inside of zone 1, and record each distance to the nearest 0.01 mm. Now calculate the arithmetic mean among all measured emboss spacings measured between nearest neighbors inside of zone 1, and record as Zone 1 Emboss Spacing to the nearest 0.01 mm.

In like fashion, the entire procedure is repeated for each additional zone containing emboss elements that is present on the test sample and label accordingly as Zone 2, Zone 3, etc.

### Nonwoven Thickness—Pressure Method

The thickness of a test specimen is measured as the distance between a reference platform on which the specimen rests and a pressure foot that exerts a specified amount of pressure onto the specimen over a specified amount of time. For purposes herein, thickness is measured and reported at two different confining pressures (7 g/cm² and 70 g/cm²). All measurements are performed in a laboratory maintained at 23° C.±2° C. and 50%±2% relative humidity and test specimens are conditioned in this environment for at least 2 hours prior to testing.

Thickness is measured with a manually-operated micrometer equipped with a pressure foot capable of exerting a steady pressure (7 g/cm² and 70 g/cm²) onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained from an area free of folds or wrinkles, and it must be larger than the pressure foot.

To measure thickness at a confining pressure of 7 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 7 g/cm² and report as Thickness at 7 g/cm² to the nearest 0.01 mm.

To measure thickness at a confining pressure of 70 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 70 g/cm² and report as Thickness at 70 g/cm² to the nearest 0.01 mm.

### Width, Length and Area Measurement Method

Simple dimensions like width, length, and area of specified locations (described herein) on the surface of an absorbent article are measured on images of the absorbent article sample (or prepared test specimen of a given zone) acquired using a flatbed scanner. The scanner is capable of scanning in reflectance mode at a resolution of 2400 dpi and 8 bit grayscale. A suitable scanner is an Epson Perfection V750 Pro from Epson America Inc., Long Beach CA, or equivalent. The scanner is interfaced with a computer running an image analysis program. A suitable program is ImageJ v. 1.52, National Institute of Health, USA, or equivalent. The sample images are distance calibrated against an acquired image of a ruler certified by NIST. To enable maximum contrast, the specimen is backed with an opaque, black background of uniform color prior to acquiring the image. All testing is performed in a conditioned room maintained at about 23±2° C. and about 50±2% relative humidity.

The test sample is prepared as follows. Remove the absorbent article from any wrapper present. If the article is folded, gently unfold it and smooth out any wrinkles. If wings are present, extend them but leave the release paper intact. In like fashion, a total of five replicate intact test samples are prepared. The test samples are conditioned at about 23° C.±2° C. and about 50%±2% relative humidity for 2 hours prior to testing. To note, the area of test specimens prepared as specified in the basis weight section of the Absorbent Article Caliper, Basis Weight, and Density Method, described herein, is also measured using this imaging technique, and no further preparation of those test specimens is required.

Images are obtained as follows. The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and the test sample, or test specimen, is scanned as follows. Place the test sample, or prepared test specimen, onto the center of the scanner glass and secure, if necessary, such that it lies flat with the body-facing surface of the test sample, or prepared test specimen, facing the scanner's glass surface. The test sample, or prepared test specimen, is oriented in such a way that the entire test sample, or prepared test specimen, is within the glass surface. The black background is placed on top of the test sample, or prepared test specimen, the scanner lid is closed, and a scanned image of the entire test sample, or prepared test specimen, is acquired with the same settings as used for the calibration image. The test sample, or prepared test specimen image is saved as an uncompressed TIFF format file.

The test sample image is analyzed to make width and length measurements as follows. Open the calibration image file in the image analysis program, and calibrate the image resolution using the imaged ruler to determine the number of pixels per millimeter. Now open the test sample image in the image analysis program, and set the distance scale using the image resolution determined from the calibration image. Linear measurements are made using the line measurement tool within the image analysis software. As depicted in FIG. 2 and FIG. 4, widths WS, W1, W2 and W3 and lengths LC and LT are measured using the locations for each dimension as specified herein. A total of five replicate test samples are imaged and analyzed in like fashion, and each linear parameter is recorded to the nearest 0.1 mm. The arithmetic mean among the five replicate test samples is calculated for each of the linear parameters and reported as WS, W1, W2, W3, LC, and LT to the nearest 0.1 mm.

The test specimen images of the central absorbent zone and outer absorbent zone are analyzed as follows. Open the calibration image file in the image analysis program, and calibrate the image resolution using the imaged ruler to determine the number of pixels per millimeter. Now open the test specimen image in the image analysis program, and set the distance scale using the image resolution determined from the calibration image. Area measurements are made using the freehand selection tool, or equivalent, within the image analysis software to draw a shape that runs along the outer perimeter of the prepared test specimen. Now measure the area of the drawn shape and record as area to the nearest 0.01 mm², denoting the zone as either central or outer absorbent zone, and also denoting the corresponding sample number. In like fashion, repeat until the area of each of the five replicate test specimens from each zone (central and outer absorbent zones) is measured and recorded, denoting the corresponding sample number for each replicate. Now proceed back to the basis weight portion of the Absorbent Article Caliper, Basis Weight, and Density Method.

### Absorbent Article Caliper, Basis Weight, and Density Method

The caliper, basis weight, and density method specifies how these parameters are measured for two different test locations on an absorbent article sample. Referring to FIG. 2, the test locations include the central absorbent zone 306 and the outer absorbent zone 325. All testing is performed in a room controlled at 23° C.±3° C. and 50%±2% relative humidity.

The absorbent article test samples are conditioned at 23° C.±3° C. and 50%±2% relative humidity two hours prior to testing. Remove the test sample from its outer wrapper, then remove the protective cover/release paper from the panty fastening adhesive on the garment facing side of the sample. Lightly dust the panty fastening adhesive with talc powder to mitigate any tackiness. In like fashion, a total of five replicate test samples are prepared. The test samples are labeled consecutively as sample 1 through sample 5 by marking a small number on the backsheet/garment side of each sample.

### Absorbent Article Caliper

Caliper (or “thickness”) is measured at specified test locations on the absorbent article sample using a manually-operated micrometer equipped with a pressure foot capable of exerting a steady pressure of 7 g/cm². The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test sample is supported by a horizontal flat reference platform that is larger than and parallel to the surface of the pressure foot. Zero the micrometer against the horizontal flat reference platform. Place the test sample onto the platform, with the test location centered beneath the pressure foot. The pressure foot is lowered by hand with a descent rate of 3±1 mm/s until the full weight of the pressure is exerted onto the sample. After 5 seconds elapse, the thickness is recorded as absorbent article caliper to the nearest 0.01 mm, denoting the test location as either central or outer absorbent zone, and also denoting the sample number, as previously marked. As specified, the caliper is measured at three separate, non-overlapping regions within the central absorbent zone 306 of each intact absorbent article sample. The arithmetic mean of the caliper values collected for the central absorbent zone 306 across all five replicate samples is calculated and reported as Absorbent Article Caliper in the Central Absorbent Zone to the nearest 0.01 mm. Now the caliper is measured at three separate, non-overlapping regions within the outer absorbent zone 325 of each intact absorbent article sample. The arithmetic mean of the caliper values collected for the outer absorbent zone 325 across all five replicate samples is calculated and reported as Absorbent Article Caliper in the Outer Absorbent Zone to the nearest 0.01 mm. Proceed to the basis weight measurement using the same prepared and numbered test samples.

### Absorbent Article Basis Weight

The basis weight of the central absorbent zone 306 and the outer absorbent zone 325 are measured separately for each prepared test sample using specially machined cutting dies. A first cutting die in the exact shape of the outer absorbent zone is prepared such that the cutting line of the die is aligned with the exact perimeter of the entire region designated as the outer absorbent zone 325, as depicted in FIG. 2. A second cutting die in the exact shape of the central absorbent zone is prepared such that the cutting line of the die is aligned with the exact perimeter of the entire region designated as the central absorbent zone 306, as depicted in FIG. 2. It is understood that for each unique product design, and for every different product size, a set of specially machined cutting dies must be prepared. Measure the mass of the central absorbent zone 306 and the outer absorbent zone 325 on the test sample as follows. Align the specially machined first cutting die with the perimeter of the outer absorbent 325, and punch out a first test specimen that includes both the outer and central absorbent zones. Now align the specially prepared second cutting die with the perimeter of the central absorbent zone 306 on the first test specimen, and punch out a second test specimen that includes only the central absorbent zone 306, with the remaining portion of the first test specimen now comprising only the outer absorbent zone 325. The corresponding sample number is labeled on the backsheet/garment side of each of these prepared test specimens. Careful handling of the test specimens is required to prevent the loss of any particles or material fibers contained within each. Record the mass of the specimen that is the central absorbent zone, and record as absorbent article central absorbent zone mass to the nearest 0.001 g, denoting the sample number as well. Now record the mass of the specimen that is the outer absorbent zone, and record as absorbent article outer absorbent zone mass to the nearest 0.001 g, denoting the sample number as well. In like fashion, punch out the central and outer absorbent zones, labeling the corresponding sample number one each, for the remaining four replicate test samples. Record the mass of each zone to the nearest 0.001 g, denoting the zone as either absorbent article central or outer absorbent zone, and also denoting the corresponding sample number. Now proceed to the Width, Length and Area Measurement Method, as described herein, to measure the area of each of the test specimens representing the absorbent article central and outer absorbent zones. After obtaining the area of each zone, calculate the basis weight by dividing the mass (g) by the area (converted to square meters), and record as absorbent article basis weight to the nearest 0.1 g/m², denoting the zone as either central or outer absorbent zone, and also denoting the corresponding sample number. Calculate the arithmetic mean of the basis weight across all five test specimens of the central absorbent zone, and report as Absorbent Article Basis Weight of the Central Absorbent Zone to the nearest 0.1 g/m². Calculate the arithmetic mean of the basis weight across all five test replicates of the outer absorbent zone, and report as Absorbent Article Basis Weight of the Outer Absorbent Zone to the nearest 0.1 g/m². Retain all of these prepared test specimens of the absorbent article central and outer absorbent zones as they will be used for the Basis Weight of the Inner Core Layer method, as described herein.

### Absorbent Article Density

The density of the central absorbent zone 306 and the outer absorbent zone 325 are calculated as follows. For the central absorbent zone test specimen from Sample 1, divide the basis weight in g/m² (absorbent article basis weight, central absorbent zone, sample 1) by the caliper, mm (absorbent article caliper, central absorbent zone, sample 1), then divide the quotient by 1000 and record as absorbent article density of the central absorbent zone to the nearest 0.001 g/m³. In like fashion, calculate density of the central absorbent zone for all five replicate test specimens. Now calculate the arithmetic mean of density across all five test specimens of the central absorbent zone, and report as Absorbent Article Density of the Central Absorbent Zone to the nearest 0.001 g/m³. For the outer absorbent zone test specimen from Sample 1, divide the basis weight in g/m² (absorbent article basis weight, outer absorbent zone, sample 1) by the caliper, mm (absorbent article caliper, outer absorbent zone, sample 1), then divide the quotient by 1000 and record as absorbent article density of the outer absorbent zone to the nearest 0.001 g/m³. In like fashion, calculate density of the outer absorbent zone for all five replicate test specimens. Now calculate the arithmetic mean of density across all five test specimens of the outer absorbent zone, and report as Absorbent Article Density of the Outer Absorbent Zone to the nearest 0.001 g/m³.

### Inner Core Layer Basis Weight

The basis weight of the inner core layer, at the points in the central absorbent zone and the outer absorbent zone will typically be known by the manufacturer from the product making specification. However, if the basis weight is not known for a given article, the basis weight can be measured in the following manner.

The basis weight of the inner core layer within the central absorbent zone 306 and the outer absorbent zone 325 are measured separately for each of the retained absorbent article test specimens that were prepared in the basis weight section of the Absorbent Article Caliper, Basis Weight, and Density Method, as described herein. There will be five absorbent article test specimens that are the central absorbent zone (labeled as samples 1-5) and five absorbent article test specimens that are the outer absorbent zone (also labeled as samples 1-5). To note, the measured area values for each test specimens that were previously recorded to calculate the basis weight of the absorbent article central and outer absorbent zones in the basis weight section of the Absorbent Article Caliper, Basis Weight, and Density Method will also be used to calculate the basis weight of the inner core layer from each of these zones.

The inner core layer is removed from the test specimens, the mass recorded, and the basis weight calculated as follows. Begin with the absorbent article test specimen of the central absorbent zone from sample 1. Carefully remove the topsheet layer, backsheet film, the upper nonwoven layer and the lower nonwoven layer from the inner core layer of the test specimen, ensuring that in the process no particles or fibers are lost from the inner core layer. It is understood that, depending on the unique structure of the absorbent article, additional layers may need to be removed from the absorbent article test specimen in order to obtain a final test specimen that comprises only the inner core layer. Record the mass of the inner core layer test specimen to the nearest 0.001 g, denoting central absorbent zone, sample 1. Divide the mass of the inner core layer from the central absorbent zone of sample 1 by the area of the absorbent article central absorbent zone of sample 1 (previously measured), and record as basis weight of the inner core layer, denoting central absorbent zone, sample 1, to the nearest 0.1 g/m². In like fashion, repeat the procedure until the basis weight of the inner core layer from the central absorbent zone is measured and recorded for each of the five central absorbent zone test specimen replicates from samples 1 through 5. Calculate the arithmetic mean across the basis weight values obtained for all five central absorbent zone replicates, and report as Inner Core Layer Basis Weight of the Central Absorbent Zone to the nearest 0.1 g/m². In like fashion, this entire procedure is repeated for the five absorbent article test specimens from the outer absorbent zone of samples 1 through 5, and the arithmetic mean across the five basis weight values is calculated and reported as Inner Core Layer Basis Weight of the Outer Absorbent Zone to the nearest 0.1 g/m².

### Z-Compression Method

The Z-compression method measures the compression behavior along the z-direction of a test specimen, on a Constant Rate of Extension (CRE) universal mechanical test system using a load cell for which the forces measured are within 1% to 99% of the limit of the cell (preferably 100 N). A suitable instrument is the MTS Alliance using TestSuite Software, as available from MTS Systems Corp., Eden Prairie, MN, or equivalent. All testing is performed in a room controlled at 23° C.±3° C. and 50%±2% relative humidity.

The upper and lower fixtures of the test system are circular parallel plate compression platens made of stainless steel. The platen mounted on the moveable CRE fixture has a diameter of 40 mm, and the platen mounted on the stationary CRE fixture has a diameter >40 mm. Both platens have adapters compatible with the mounts of the CRE test machine, capable of securing the platens with their opposing surfaces lying along parallel planes that are orthogonal to the motion of the crossbeam of the CRE test machine.

The absorbent article samples are conditioned at 23° C.±3° C. and 50%±2% relative humidity two hours prior to testing. Remove the test sample from its outer wrapper, then remove the protective cover/release paper from the panty fastening adhesive on the garment facing side of the sample. Lightly apply talc powder to the adhesive to mitigate any tackiness. To obtain a test specimen for measurement, a circular die with a diameter of 40 mm is used. Test specimens are obtained from two separate test locations on the absorbent article test sample from areas that are free from any folds or wrinkles, as follows. Referring to FIG. 4, the first test specimen is obtained from the rear-most portion of the central absorbent zone 306, more specifically denoted as the region where W3 is measured, such that the test specimen is centered within this test location. The second test specimen is obtained from the rear-most portion of the outer absorbent zone 326, such that the test specimen is centered within this test location, inbound of the perimeter seal, and ideally comprises no portion of the central absorbent zone. In like fashion, five replicate test specimens are prepared from the central and outer absorbent zones on five replicate test samples.

Prepare the universal test frame for a compression test to measure force and distance for one cycle of loading (compression) and unloading (recovery) as follows. The crosshead motion is programmed such that the upper platen moves down with respect to the lower platen at a rate of 0.2 mm/s until an endpoint load of 8.66 N (6.9 kPa) is reached, then the crosshead immediately returned to the original gauge (platen separation distance).

Execute the test as follows. Move the platens such that the initial distance between contact surfaces of the platens (gauge) is 25 mm, then zero the crosshead and load cell. Place the test specimen, with the wearer-facing surface upward, onto the bottom platen with the longitudinal and lateral midpoint of the test specimen centered under the upper platen. Manually adjust the position of the upper platen such that its contact surface is about 1 mm above the upper surface of the test specimen. Start the test and continuously collect force (N) and displacement (mm) data at a rate of 100 Hz.

Construct a graph of force (N) versus thickness (mm), across the array of data collected for the entire cycle. To note, at each datapoint, thickness is the original gauge (25 mm) minus the crosshead position (mm). From the resulting force (N) vs thickness (mm) curve, calculate the area under the loading (compression) portion of the curve from the initial thickness to the minimum thickness, and record as energy of z-compression to the nearest 0.1 N*mm, denoting the test location as central or outer absorbent zone.

In like fashion, repeat the procedure for all five replicate test specimens from the central absorbent zone and all five replicate test specimens from the outer absorbent zone. Calculate and report the arithmetic mean across all five replicates from each test location and report as Energy of Z-Compression to the nearest 0.1 N*mm, denoting central or outer absorbent zones.

### Micro-CT Measurement Method

The micro-CT measurement method is used to obtain images of the cross-section of a test specimen to enable visualization of the microstructure of an absorbent article, including the interconnectivity of layers within the article in specific regions of interest. These images enable qualitative and quantitative assessments to be made related to the proximity of adjacent layers within the test specimen, and the resultant size and shape of specified zones located within the test specimen. This method is based on analysis of a 3D x-ray sample image obtained on a micro-CT instrument (a suitable instrument is the Scanco μCT 50 available from Scanco Medical AG, Switzerland, or equivalent). The micro-CT instrument is a cone beam microtomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and reconstruction of the raw data into a 3D image. The 3D image is then analyzed using image analysis software (suitable image analysis software are MATLAB available from The Mathworks, Inc., Natick, MA, and Avizo 2022.2 available from Visualization Sciences Group/FEI Company, Burlington, MA, or equivalents) to identify specified zones with the test specimen, measure the distances between individual layers and zones, thickness of zones as well as any angle created as one zone transitions to another within the test specimen.

### Sample Preparation:

A test specimen is excised from the test sample using a very sharp blade as follows. The test specimen is taken from an area free of folds or wrinkles using care to not impart any contamination or distortion to the specimen during the preparation process. The test specimen is excised from a region of the test sample in such a way that it comprises a portion of both the central absorbent zone and the outer absorbent zone, including any transition zone that may be present. The test specimen is roughly 90 mm in diameter. In like fashion, a total of 3 replicate test specimens are prepared from three different test samples. The test specimens are conditioned at about 23° C.±2° C. and about 50%±2% relative humidity for 2 hours prior to testing.

### Image Acquisition:

The micro-CT instrument is set up and calibrated according to the manufacturer's specifications. The test specimen is placed on a low density foam and placed into the appropriate holder. This will allow the test specimen to lay horizontal and be scanned with minimal attenuation from any surrounding material. A single 3D dataset of contiguous 13 μm (microns) isotropic voxels is collected. The 3D data set has dimensions of 96.7 mm on each side in the XY-plane and a sufficient number of slices to fully include the entire Z-direction of the test specimen. Images are acquired with the source at 70 kev and 114 μA with no additional low energy filter. These current and voltage settings may be optimized to produce the maximum contrast in the projection data with sufficient x-ray penetration through the test specimen, but once optimized held constant for all substantially similar test specimens. A total of 3000 projection images are obtained with a total integration of 500 msec integration time and 4 averages per projection. The projection images are reconstructed into a 3D dataset having an isotropic spatial resolution of 13 μm (microns), and saved in 16-bit RAW format to preserve the full detector output signal for analysis. For optimal visualization purposes, the data was scaled to 8 bit using a scale factor of 0.4 and subsampled to 26 micron resolution.

### Image Processing:

The 3D dataset is loaded into the image analysis software and trimmed (cropped) to a rectangular prism 3D image of the analysis region by removing the surrounding holder and the low density mounting material from the 3D dataset. Trimming is performed such that the maximum amount of the test specimen in the analysis region is retained in the 3D image, and the empty space above and below the test specimen is minimized. Within the 3D image, every 10 cross sectional slices are averaged together to create less noise. This averaging creates a thicker slice representing a 260 micron thick slab along the viewing direction. In-plane resolution is 26 microns.

The 3D image is oriented so that the upper surface (topsheet, or body size of the test specimen) is as close to parallel with the XY-plane as possible. Now qualitative observations can be made regarding the proximity of adjacent layers present in the test specimen including interconnectivity between said layers and the overall shape of the various zones present (i.e. central and outer absorbent zones and the transition between said zones). In addition to the qualitative observations, simple quantitative measures (e.g. thickness of zones, distance between zones, the angle formed as one zone transitions to the other, etc.) are possible using measurement tools available within the image analysis software.

### Examples/Data

The following data and examples, including comparative examples, are provided to help illustrate the upper and lower nonwoven layers and/or absorbent articles described herein. The exemplified structures are given solely for the purpose of illustration and are not to be construed as limitations of the present disclosure, as many variations thereof are possible without departing from the spirit and scope of the invention.

### Nonwoven Layer Material Test

A series of measurements were performed on nonwoven materials to assess the ability of the material to function as an upper nonwoven layer and/or a lower nonwoven layer in the absorbent core structure described herein. Samples A-G are examples in accordance with the present disclosure. Comparative Sample H is a comparative example. Samples A-G and Comparative Sample H are described in Table 1 below.

Samples A-G and Comparative Sample H were evaluated according to the CD Cyclic Elongation to 3% Strain Method, the Strain to Break Method, Dry CD Ultra Sensitive 3 Point Bending Method, and the Nonwoven Thickness-Pressure Method. The results are shown in Table 2.

TABLE 1
Nonwoven Material Description

| Sample | Nonwoven Material | Fiber Composition |
|---|---|---|
| A | 40 gsm Carded | BiCo (PE/PET)—60% 2 DTex/ |
| | Resilient | 40% 4 DTex Blend |
| | Nonwoven¹ | |
| B | 55 gsm Resilient | 30% 10 DTex |
| | Spunlace 1² | HS-PET; |
| | | 20% 1.3 DTex Rayon; |
| | | 50% 2.2 DTex BiCo (PE/PET) |
| C | 50 gsm Resilient | 20% 1.3 DTex Rayon; |
| | Spunlace 6³ | 20% 3.3 DTex tri-lobal Rayon; |
| | | 60% 5.8 DTex PE/PET |
| D | 24 gsm Carded | 100% 2 DTex BiCo (PE/PET) |
| | Nonwoven⁴ | |
| E | 55 gsm Resilient | 40% 1.7 DTex/38 mm Rayon; |
| | Spunlace 5⁵ | 40% 2.2 DTex PET; |
| | | 20% 10 DTex HS PET |
| F | 18 gsm Spunbond | 100% 2.0 DTex PP |
| | Nonwoven⁶ | |
| G | 25 gsm Spunbond | 100% 2.0 Dtex PP |
| | Nonwoven⁷ | |
| Comparative | 17 gsm Tissue⁸ | 100% Cellulose |
| Sample H | | |

¹Available as ATB Z87G-40 from Xiamen Yanjan New Material Co. (China)
²Available as Sawasoft ® 53FC041001 from Sandler GmbH (Germany)
³Available as Sawasoft ® 553FC041005 (option 82) from Sandler GmbH (Germany)
⁴Available as Aura 20 from Xiamen Yanjan New Material Co. (China)
⁵Available as S25000541R01 from Jacob Holms Industries (Germany)
⁶Available as PFNZN 18G BICO8020 PHI 6 from dPFNonwovens Czech S.R.O (Czech Republic)
⁷Available as PEGZN25 BICO7030 Phobic from dPFNonwovens Czech S.R.O (Czech Republic)
⁸Available as 3028 from DunnPaper (USA)

TABLE 2

| | | | Dry | Dry | | | |
|---|---|---|---|---|---|---|---|
| | Permanent | % Strain | Bending | Recovery | Density | Thickness | Thickness |
| | Strain | to Break | Energy | Energy | at 7 g/cm² | at 7 g/cm² | at 70 g/cm² |
| Sample | mm/mm | % | N*mm | N*mm | g/cm³ | mm | mm |
| A | 0.006 | >10% | 0.219 | 0.092 | 0.03 | 1.21 | 0.26 |
| B | 0.006 | >10% | 1.015 | 0.291 | 0.06 | 0.99 | 0.44 |
| C | 0.005 | >10% | 0.595 | 0.201 | 0.07 | 0.76 | 0.62 |
| D | 0.016 | >10% | 0.176 | 0.036 | 0.05 | 0.45 | 0.19 |
| E | 0.013 | >10% | 0.059 | 0.032 | 0.07 | 0.80 | 0.40 |
| F | 0.010 | >10% | 0.022 | 0.005 | 0.09 | 0.21 | 0.17 |
| G | 0.009 | >10% | 0.062 | 0.019 | NA | NA | NA |
| Comp. H | 0.014 | <5% | 0.073 | 0.031 | 0.12 | 0.14 | 0.11 |

It is believed that nonwoven materials suitable for upper and/or lower nonwoven layers can strain (elongate) with a balanced stretch and recover to their original state, thus helping to enable the absorbent core structure and/or absorbent article to recover from deformation during bodily motions. The 3D inner core layer shape described herein is configured to fit closely to the wearer's intimate genitalia and rest between the labial majora. As such, particularly suitable nonwoven materials of the upper nonwoven layer can provide fluid handling performance that can effectively transport fluid deep into the inner core layer to help provide a close and comfortable fit to the body that feels dry. To achieve this, suitable nonwoven materials of the upper nonwoven layer exhibit a relatively low density (e.g., from 0.03 to 0.07 g/cm³ under a pressure of 7 g/cm²) in order to allow fluid to efficiently drain from the upper nonwoven layer into the inner core layer below. In addition, suitable nonwoven materials of the upper nonwoven layer can maintain a relatively lofty thickness even under high bodily compressive forces (i.e., a pressure of 70 g/cm²) so that fluid residing within the inner core layer is not expelled back out of the absorbent core structure which can create a wet feeling on the body. Nonwoven materials suitable for upper and/or lower nonwoven layer may also need to be able to follow and assume the complex 3D inner core layer shapes described herein.

It was found that Samples A-C and E are suitable materials for upper and/or lower nonwoven layers. In particular, Samples A-C and E had a Permanent Strain of 0.013 mm/mm or less, demonstrating that the materials can elongate and recover, and had a Strain to Break of greater than 10% before tearing. Samples A-C and E also required less energy to bend (demonstrated by a Dry Bending Energy of less than 1.6 N*mm) while recovering from bending with a Dry Recovery Energy of greater than 0.03 N*mm. Samples A-C and E exhibited a relatively low density of from 0.03 to 0.07 g/cm³ under a pressure of 7 g/cm² and a Thickness at 7 g/cm² of pressure of from 0.80 to 1.21 mm, demonstrating that these materials are both lofty and have a more open fiber network structure that can help with efficient fluid handling performance.

It was found that Sample D had a Permanent Strain of 0.016 mm/mm, demonstrating that the material will likely elongate strongly during manufacture and/or in-use without recovering to its initial state. As such, it is believed that the material may not be capable of maintaining the structural stability and shape of the inner core layer. At the same time, Sample D was found to be highly compressible under bodily pressures, as demonstrated by a Thickness at 70 g/cm² of 0.19 mm, illustrating that the material will become more dense under bodily compression and likely will not be sufficiently drained of fluid by the inner core layer. Samples F and G exhibited a Dry Recovery Energy of less than 0.03 N*mm, demonstrating that the materials will not recover from deformation, making the material insufficient to function as an upper nonwoven layer. However, when combined with an upper nonwoven layer described herein, Samples D, F, and G are suitable materials for a lower nonwoven layer.

Comparative Sample H exhibited a Strain to Break of less than 5% and a Thickness at 70 g/cm2 of less than 0.2 mm. In addition, it was found that Comparative Sample H tears when wet. As such, Comparative Sample H is insufficient to function as an upper or lower nonwoven layer.

Table 3 is provided for the convenience of the reader. Table 3 includes a non-exhaustive list of properties as well as a non-exhaustive list of corresponding values for each of the properties that particularly suitable upper nonwoven layers of the present disclosure may exhibit.

TABLE 3
Upper Nonwoven Layer

| Property | Value |
|---|---|
| Permanent Strain | 0.005 to 0.013 mm/mm |
| % Strain to Break | Greater than 10% |
| Dry Recovery Energy | Greater than 0.03 N * m |
| Density at 7 g/cm² | 0.03 to 0.07 g/cm³ |
| Thickness at 7 g/cm² | 0.3 mm to 1.3 mm |
| Thickness at 70 g/cm² | 0.2 mm to 0.7 mm |

### Finished Product Test

Absorbent articles are tested to assess the ability of the absorbent article to bend and compress in the Z-direction so as to conform to user's specific genital shape. Ex. 1-3 illustrate absorbent articles described herein. A description of Ex. 1-3 are listed in Table 4a. Ex. 1-3 are prepared as described hereafter. The absorbent articles are evaluated according to the Dry MD 3 Point Bend Method, Absorbent Article Caliper, Basis Weight and Density Method, and the Z-Compression Method as described herein. The results are reported as averages in Table 4b.

TABLE 4a
Absorbent Article Description

| | | Absorbent core structure | | |
|---|---|---|---|---|
| | | Upper | Inner Core Layer | Lower |
| | | Nonwoven | (Total Basis | Nonwoven |
| Example | Topsheet | Layer | Weight) | Layer |
| Ex. 1 | 24 gsm Carded | 40 gsm Carded | 224 gsm Fluff¹¹/ | 18 gsm |
| | Nonwoven⁹ | Resilient | 89 AGM¹² | Spunbond |
| | | Nonwoven¹ | | Nonwoven⁶ |
| Ex. 2 | 24 gsm Carded | 40 gsm Carded | 241 gsm Fluff¹¹/ | 18 gsm |
| | Nonwoven¹⁰ | Resilient | 97 gsm AGM¹² | Spunbond |
| | | Nonwoven¹ | | Nonwoven⁶ |
| Ex. 3 | 24 gsm Carded | 40 gsm Carded | 176 gsm Fluff¹¹/ | 18 gsm |
| | Nonwoven¹⁰ | Resilient | 69 gsm AGM¹² | Spunbond |
| | | Nonwoven¹ | | Nonwoven⁶ |

¹Available as ATB Z87G-40 from Xiamen Yanjan New Material Co. (China)
⁶Available as PFNZN 18G BICO8020 PHI 6 from PFNonwovens Czech S.R.O (Czech Republic)
⁹Available as R73B from Xiamen Yanjan New Material Co. (China)
¹⁰Available as Z73P-24 from Xiamen Yanjan New Material Co. (China)
¹¹Available as Item 9E3-COOSABSORB S from Resolute Alabama (USA)
¹²Available as Acqualic L705 from Nippon Shokubai (Japan)

The absorbent articles listed in Table 4a were produced as detailed within the specification. Ex. 1-3 have the central absorbent zone and outer absorbent zone as shown and described in FIG. 4, with the basis weight of the inner core layer as follows: Ex. 1—Central Absorbent Zone: 256 gsm Fluff and 102 gsm AGM; Outer Absorbent Zone: 206 gsm Fluff and 82 gsm AGM gsm; Ex. 2—Central Absorbent Zone: 279 gsm Fluff and 112 gsm AGM; Outer Absorbent Zone: 220 gsm Fluff and 88 gsm AGM; and Ex. 3—Central Absorbent Zone: 200 gsm Fluff and 80 gsm AGM; Outer Absorbent Zone: 160 gsm Fluff and 62 gsm AGM.

Specifically, the upper nonwoven layer is first introduced onto the forming drum within the laydown section, and under vacuum it is drawn into the 3 dimensional pocket shape. A homogeneous stream of the cellulose and superabsorbent particle material is deposited onto the upper nonwoven layer directly within the forming station. Prior to entering the forming station, the upper nonwoven is coated with a spray adhesive (Technomelt DM 9036U available from Henkel, (Germany), 6 gsm continuous meltblown spirals, 50 mm wide) to provide a stronger connection of the cellulose and superabsorbent particle material to the upper nonwoven layer without hindering the flow of liquid into the cellulose and superabsorbent particle material. On exiting the laydown section, the lower nonwoven web is combined with the nonwoven carrying the homogeneous blend of cellulose and superabsorbent particle material. This lower nonwoven is precoated with adhesive (Technomelt DM 9036U available from Henkel (Germany)) to enable a perimeter seal (10 gsm meltblown spirals, 20 mm wide on the sides) and in the center a 6 gsm, 50 mm wide continuous meltblown spiral adhesive (Technomelt DM 9036U available from Henkel (Germany)) is applied to better integrate the cellulose and superabsorbent particle material. Excess nonwoven material beyond the perimeter seal is removed prior to addition of the topsheet and the backsheet. The topsheet is bonded to the absorbent core structure with a spray adhesive application (Technomelt DM 9036U available from Henkel (Germany), 3 gsm continuous meltblown spirals, 50 mm wide, 250 mm long). In addition, a 12 gsm polypropylene backsheet is bonded to the outward-facing surface of the lower nonwoven with a spray adhesive application (Technomelt DM 9036U available from Henkel (Germany), 3 gsm continuous meltblown spirals, 50 mm wide, 250 mm long).

Ex. 1-3 also have the structural bonds as shown in FIGS. 4 and 5 with the profile shown in FIG. 6. The structural bonds are applied with a heated aluminum die to create an emboss pattern within a heated hydraulic press. The structural bond embosser plate has protrusions of an area of 3.55 mm² and about 1 mm in height as shown in FIG. 5 with the profile shown in FIG. 6. The structural bonds are spaced according to the dimensions of separation described above. The structural bond embosser plate is heated to 120° C. and set to a compression pressure of 170 kPa. The absorbent article is placed and orientated underneath the heated embosser plate on the hydraulic press bottom plate and a sheet of thin Teflon™ film is placed over the sample prior to embossing to avoid melting of the topsheet fibers. The hydraulic press is activated and compresses the sample for a dwell time of 1.7 seconds to create the structural bond pattern.

Prior to bonding the backsheet, flex bond channel regions are applied to Ex. 1-3 with the pattern shown in FIG. 9. The flex bond channel regions are applied with a heated aluminum die to create an emboss pattern within a heated hydraulic press. The channel embosser plate has protrusions spaced about 1.5 mm apart and are about 3 mm long and about 1.5 mm wide. The bond channel embosser plate is heated to 120° C. and set to a compression pressure of 200 kPa. The absorbent article is placed and orientated underneath the heated embosser plate on the hydraulic press bottom plate and a sheet of thin Teflon™ film is placed over the sample prior to embossing to avoid melting of the topsheet fibers. The hydraulic press is activated and compresses the sample for a dwell time of 1.7 seconds to create the emboss pattern.

TABLE 4b
Absorbent Articles Tested in the Dry MD 3 Point Bend Method, Absorbent Article
Caliper, Basis Weight and Density Method, and the Z-Compression Method

| | Dry MD 3 Point Bend Method & Absorbent Article | | | | | |
|---|---|---|---|---|---|---|
| | Caliper, Basis Weight and Density Method | | | | | |
| | | | Outer Absorbent | | Z-Compression Method | |
| | Central Absorbent Zone | | Zone | | Central | Outer Absorbent |
| | | MD Dry | | MD Dry | Absorbent Zone | Zone |
| | | Bending | | Bending | Energy of | Energy of |
| | Caliper | Modulus | Caliper | Modulus | Z-Compression | Z-Compression |
| Example | (mm) | (N/mm²) | (mm) | (N/mm²) | (N · mm) | (N · mm) |
| Ex. 1 | 4.24 | 0.101 | 2.84 | 0.132 | 5.1 | 3.7 |
| Ex. 2 | 4.31 | 0.085 | 2.86 | 0.081 | 6.3 | 4.4 |
| Ex. 3 | 3.09 | 0.117 | 2.53 | 0.159 | 4.5 | 3.8 |

Ex. 1-3 exhibited a Caliper in the central absorbent zone of from 3.09 mm to 4.24 mm and a Caliper in the outer absorbent zone of from 2.53 mm to 2.86 mm. It is believed that in order to create an absorbent article that fits closely and comfortably in the outer space between the labial majora, the caliper of the central absorbent zone should be sufficient to fit close to the body with the caliper of the outer absorbent zone being thinner to allow the sides to gently envelope around the left and right outer side surfaces of the labial majora. Ex. 1-3 exhibited a low MD Bending Modulus, demonstrating that the central absorbent zone and the outer absorbent zone are highly flexible and can bend and adapt to a variety of genital shapes with low bending resistance and thus provide more comfort. Ex. 1-3 also exhibited an Energy of Z-Compression in the central absorbent zone and the outer absorbent zone of from 3.8 to about 4.4 N·mm, demonstrating that the absorbent article has a high compressibility in the Z-plane. Ease of bending (low modulus) and high Z-compressibility allows for the raised central absorbent zone to first adapt and mold to her intimate shape but also to deform and compress with the range of bodily motions such as walking, sitting or exercise without discomfort.

### Combinations/Examples

Paragraph A. A disposable absorbent article comprising:
a topsheet;
a backsheet; and
   an absorbent core structure disposed between the topsheet and the backsheet, wherein the absorbent core structure comprises:
a. an upper nonwoven layer comprising polymer fibers and having a basis weight of from about 35 gsm to about 85 gsm;
b. a lower nonwoven layer comprising polymer fibers and having a basis weight of from about 10 gsm to about 40 gsm; and
c. an inner core layer disposed between the upper nonwoven layer and the lower nonwoven layer, wherein the inner core layer comprises a mixture of cellulosic fibers and superabsorbent particles;
wherein the inner core layer further comprises a central absorbent zone having a first basis weight and an outer absorbent zone having a second basis weight; wherein the outer absorbent zone substantially surrounds the central absorbent zone; wherein the first basis weight is greater than the second basis weight;
wherein the inner core layer is a unitary structure;
wherein the absorbent article has a first average density measured in the central absorbent zone and a second average density measured in the outer absorbent zone, wherein the second density is within about 0 to about 20% of the first density.
   Paragraph B. The disposable absorbent article of Paragraph A, wherein the absorbent article has a caliper measured in the central absorbent zone of from about 2.5 mm to about 6 mm.
   Paragraph C. The disposable absorbent article of Paragraph A or B, wherein the first and second average density are between about 0.045 g/cm³ and about 0.150 g/cm³.
   Paragraph D. The disposable absorbent article of Paragraphs A to C, wherein the central absorbent zone comprises a pair of inwardly concave longitudinal side edges, an outwardly convex front edge, and an outwardly convex rear edge.
   Paragraph E. The disposable absorbent article of Paragraphs A to D, wherein the central absorbent zone comprises a front region having a first lateral width W1, a rear region having a third lateral width W3, and a middle region disposed therebetween having a second lateral width W2, wherein the second lateral width W2 is less than at least one of the first lateral width W1 and the third lateral width W3.
   Paragraph F. The disposable absorbent article of Paragraph E, wherein the third lateral width W3 is greater than the first lateral width W1 and the second lateral width W2.
   Paragraph G. The disposable absorbent article of Paragraphs A to F, wherein the absorbent article exhibits an Energy of Z-Compression measured in the central absorbent zone and the outer absorbent zone of from about 3.0 to about 8.0 N·mm.
   Paragraph H. The disposable absorbent article of Paragraphs A to G, wherein the absorbent article exhibits a MD Bending Modulus measured in the central absorbent zone and the outer absorbent zone of from about 0.03 to about 0.18 N/mm².
   Paragraph I. The disposable absorbent article of Paragraphs A to H, wherein the central absorbent zone comprises a transition zone wherein the basis weight of the inner core layer gradually decreases; the transition zone comprising a width of from about 1 mm to about 5 mm.
   Paragraph J. The disposable absorbent article Paragraphs A to I, wherein the absorbent core structure comprises a plurality of structural bond sites, wherein the structural bond sites have a bond area of from about 2 mm² to about 5 mm², and wherein the total structural bond area of the absorbent core structure is from about 1% to about 4% of the absorbent core structure as measured according to the Structural Bond Sites Pattern Spacing and Area Measurement Method.
   Paragraph K. The disposable absorbent article of Paragraph J, wherein the average distance between the structural bond sites is from about 10 mm to about 32 mm as measured according to the Structural Bond Sites Pattern Spacing and Area Measurement Method.
   Paragraph L. The disposable absorbent article of Paragraphs A to K, wherein the polymer fibers of the upper nonwoven layer have a length of from about 10 mm to about 100 mm.
   Paragraph M. The disposable absorbent article of Paragraphs A to L, wherein the polymer fibers of the upper nonwoven layer have a fiber diameter of from about 2.0 Dtex to about 10 Dtex.
   Paragraph N. The disposable absorbent article of Paragraphs A to M, wherein the upper nonwoven layer has a Permanent Strain of from about 0.005 to about 0.013 mm/mm.
   Paragraph O. The disposable absorbent article of Paragraphs A to N, wherein the upper nonwoven layer has a Thickness at 70 g/cm² pressure of from about 0.2 mm to about 0.7 mm as measured according to the Thickness—Pressure Method.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a topsheet;
a backsheet; and
an absorbent core structure disposed between the topsheet and the backsheet, wherein the absorbent core structure comprises:
a. an upper nonwoven layer comprising polymer fibers and having a basis weight of from about 30 gsm to about 85 gsm;
b. a lower nonwoven layer comprising polymer fibers and having a basis weight of from about 10 gsm to about 40 gsm; and
c. an inner core layer disposed between the upper nonwoven layer and the lower nonwoven layer, wherein the inner core layer comprises from about 50% to about 85% cellulosic fibers, by weight of the inner core layer, and from about 15% to about 50% superabsorbent particles, by weight of the inner core layer;
wherein the inner core layer is profiled in a longitudinal and a lateral direction defined by a central absorbent zone and an outer absorbent zone substantially surrounding the central absorbent zone, wherein the central absorbent zone has a first basis weight and the outer absorbent zone has a second basis weight, wherein the first basis weight is greater than the second basis weight;
wherein the absorbent article has a first average density measured in the central absorbent zone and a second average density measured in the outer absorbent zone, wherein the first and second average density are between about 0.045 g/cm³ and about 0.150 g/cm³, and wherein the second density is within about 0 to about 20% of the first density.

2. The disposable absorbent article of claim 1, wherein the inner core layer is a unitary structure.

3. The disposable absorbent article of claim 1, wherein the central absorbent zone comprises a pair of inwardly concave longitudinal side edges, an outwardly convex front edge, and an outwardly convex rear edge.

4. The disposable absorbent article of claim 1, wherein the central absorbent zone comprises a front region having a first lateral width W1, a rear region having a third lateral width W3, and a middle region disposed therebetween having a second lateral width W2, wherein the second lateral width W2 is less than the first lateral width W1 and the third lateral width W3.

5. The disposable absorbent article of claim 4, wherein the third lateral width W3 is greater than the first lateral width W1 and the second lateral width W2.

6. The disposable absorbent article of claim 1, wherein the absorbent core structure comprises a plurality of structural bond sites, wherein the structural bond sites have a bond area of from about 2 mm² to about 5 mm², and wherein the total structural bond area of the absorbent core structure is from about 1% to about 4% of the absorbent core structure as measured according to the Structural Bond Sites Pattern Spacing and Area Measurement Method.

7. The disposable absorbent article of claim 6, wherein the average distance between the structural bond sites is from about 10 mm to about 32 mm as measured according to the Structural Bond Sites Pattern Spacing and Area Measurement Method.

8. The disposable absorbent article of claim 6, wherein the central absorbent zone is substantially free from the structural bonds.

9. The disposable absorbent article of claim 1, wherein the upper nonwoven layer has a Permanent Strain of about 0.005 to about 0.013 mm/mm.

10. The disposable absorbent article of claim 1, wherein the upper nonwoven layer has a Thickness at 70 g/cm² pressure of from about 0.2 mm to about 0.7 mm as measured according to the Nonwoven Thickness—Pressure Method.

11. The disposable absorbent article of claim 1, wherein the polymer fibers of the upper nonwoven layer have a length of from about 10 mm to about 100 mm.

12. The disposable absorbent article of claim 1, wherein the polymer fibers of the upper nonwoven layer have a fiber diameter of from about 2.0 Dtex to about 10 Dtex.

13. A disposable absorbent article comprising:
a topsheet;
a backsheet; and
an absorbent core structure disposed between the topsheet and the backsheet, wherein the absorbent core structure comprises:
a. an upper nonwoven layer comprising polymer fibers and having a basis weight of from about 35 gsm to about 85 gsm;
b. a lower nonwoven layer comprising polymer fibers and having a basis weight of from about 10 gsm to about 40 gsm; and
c. an inner core layer disposed between the upper nonwoven layer and the lower nonwoven layer, wherein the inner core layer comprises a mixture of cellulosic fibers and superabsorbent particles;
wherein the inner core layer further comprises a central absorbent zone having a first basis weight and an outer absorbent zone having a second basis weight; wherein the outer absorbent zone substantially surrounds the central absorbent zone; wherein the first basis weight is greater than the second basis weight;
wherein the inner core layer is a unitary structure; and
wherein the absorbent article has a first average density measured in the central absorbent zone and a second average density measured in the outer absorbent zone, wherein the second density is within about 0 to about 20% of first density.

14. The disposable absorbent article of claim 13, wherein the absorbent article exhibits a MD Bending Modulus measured in the central absorbent zone and the outer absorbent zone of from about 0.03 to about 0.18 N/mm².

15. The disposable absorbent article of claim 13, wherein the absorbent article has a caliper measured in the central absorbent zone of from about 2.5 mm to about 6 mm.

16. The disposable absorbent article of claim 13, wherein the first and second average density are between about 0.045 g/cm³ and about 0.150 g/cm³.

17. The disposable absorbent article of claim 13, wherein the central absorbent zone comprises a pair of inwardly concave longitudinal side edges, an outwardly convex front edge, and an outwardly convex rear edge.

18. The disposable absorbent article of claim 13, wherein the central absorbent zone comprises a front region having a first lateral width W1, a rear region having a third lateral width W3, and a middle region disposed therebetween having a second lateral width W2, wherein the second lateral width W2 is less than at least one of the first lateral width W1 and the third lateral width W3.

19. The disposable absorbent article of claim 13, wherein the absorbent article exhibits an Energy of Z-Compression measured in the central absorbent zone and the outer absorbent zone of from about 3.0 to about 8.0 N·mm.

20. The disposable absorbent article of claim 13, wherein the central absorbent zone comprises a transition zone wherein the basis weight of the inner core layer gradually decreases; the transition zone comprising a width of from about 1 mm to about 5 mm.
